(19) United States
(12) Patent Application Publication
(10) Pub. No.: US 2026/0258346 A1
(43) Pub. Date: Sep. 3, 2026

(54) MICROBIAL METHODS FOR CONVERSION OF LIGNOCELLULOSIC HYDROLYSATES USING OLEAGINOUS YEAST

(71) Applicant: INSEMPRA GMBH, Planegg (DE)

(72) Inventors: Shashwat VAJPEYI, Planegg (DE); Tim ZEISKE, Planegg (DE); Niharika Sanjay SINGH, Planegg (DE)

(73) Assignee: INSEMPRA GMBH, Planegg (DE)

(21) Appl. No.: 19/478,667

(22) PCT Filed: Apr. 24, 2024
(86) PCT No.: PCT/EP2024/061210

§ 371 Date: Oct. 24, 2025

(30) Foreign Application Priority Data
Apr. 25, 2023 (EP) 23169864.8

(51) Int. Cl. C12N 1/36; C12N 1/16; C12P 7/6409

(52) U.S. Cl. CPC C12N 1/36; C12N 1/16; C12P 7/6409; C12P 2203/00

(57) ABSTRACT

The present invention lies in the field of microbial fatty acid/lipid/oil production from lignocellulosic feedstock/material/biomass, in particular from lignocellulose degradation products like lignocellulose hydrolysates. The present invention relates to a method of producing an oleaginous yeast strain adapted to a lignocellulose medium. The present invention further relates to an oleaginous yeast strain adapted to a lignocellulose medium and produced by the method of the invention and to an oleaginous yeast strain adapted to a lignocellulose medium, said strain being characterized by a particular RNA expression profile (showing, for example, an activated metabolic stress response RPOS). The present invention further relates to a method of producing oleaginous yeast cells, to a method of producing fatty acids and/or lipids, and to a method of producing (vegan) nutraceuticals/nutritional supplements or petroleum replacers. The present invention further relates to microbial methods for conversion of lignocellulosic hydrolysates using oleaginous yeast, in particular for producing oleaginous yeast cells, fatty acids, lipids, oils, (vegan) nutraceuticals/nutritional supplements and/or petroleum replacers.

The present invention lies in the field of microbial fatty acid/lipid/oil production from lignocellulosic feedstock/material/biomass, in particular by oleaginous yeast cells from lignocellulose degradation products like lignocellulosic hydrolysates. The present invention relates to a method of producing an oleaginous yeast strain adapted to a lignocellulose medium. The present invention further relates to an oleaginous yeast strain adapted to a lignocellulose medium and produced by the method of the invention and to an oleaginous yeast strain adapted to a lignocellulose medium. The present invention further relates to a method of producing oleaginous yeast cells, to a method of producing oils, fatty acids and/or lipids, and to a method of producing (vegan) nutraceuticals/nutritional supplements or petroleum replacers. The present invention further relates to (microbial methods for) conversion of lignocellulosic hydrolysates by using oleaginous yeast, in particular for producing oleaginous yeast cells, fatty acids, lipids, oils, (vegan) nutraceuticals/nutritional supplements and/or petroleum replacers.

Microbial fatty acids, lipids and oils have gained much attention during the last few years due to their potential, for example, as nutraceuticals/nutritional supplements or as replacers for petroleum as a main source of fuels and chemicals. Microbial fatty acids/lipids/oils and lipid-derived molecules can, for example, be used to produce biodiesel, biokerosene, pharmaceuticals, nutraceuticals/nutritional supplements, cosmetics, lubricants, plasticizers etc.

Currently, most of the major microbial lipid producers focus on the use of glucose as the major carbon source for fatty acid/lipid/oil production. However, there is a massive disconnect: The current global glucose market is about US$45 billion, which means about 90 million metric tons (US$500/tons). This equals about 18 million metric tons of palm oil. The current global palm oil market is about US$63 Billion, which equals about 75 million metric tons. This means that if the entire world's glucose supply would be used for microbial oil production, it could only serve 24% of the global palm oil demand. Furthermore, merely the costs for glucose for producing one kg of microbial oil are about US$2.50, i.e a raw material cost of US$2,500/ton microbial lipids can be calculated.

Actually, lignocellulose is the most abundant biomass on earth, with an estimated 182 billion tons produced annually, of which 8.2 billion tons are present in the form of agricultural wastes and residues and provide the potential of being valorized into sugars (https://onlinelibrary.wiley.com/doi/pdf/10.1111/gcbb.12586). For example, sugars from lignocellulosic biomass could be generated at costs of only US$300/ton (https://www.sciencedirect.com/science/article/abs/pii/S0926669020307007; https://online library.wiley.com/doi/abs/10.1002/bbb.2170), i.e a substantial lower raw material cost of US$1500/ton microbial lipids converted to these sugars can be calculated for this alternative carbon source.

There is thus a high desire to replace commonly applied carbon sources (such as glucose (dextrose) also in microbial fatty acid/lipid/oil production and in the respective fermentation processes, in particular of oleaginous yeasts (like, for example, Rhodotorula toruloides), with waste-based materials such as lignocellulose from organic waste like straw, stalk, wood-derived waste, etc. While the use of these materials has benefits from both, a sustainability and a cost perspective, lignocellulose hydrolysates are complex organic mixtures and certain components have been reported to exhibit toxic effects on the respective microorganisms. In particular, effective cell growth and/or productivity is inhibited by lignocellulose-derived toxins in fermentation processes.

Generally, the concept of hydrolyzing lignocellulosic carbon into fermentable sugars has been widely researched. However, no commercial scale production has been successful so far, in particular in the field of fatty acid/lipid/oil production by oleaginous yeasts. One major bottleneck in this context is that the pretreatment/degradation of lignocellulosic biomass (e.g. cell wall disruption and/or hydrolysis) also generates additional biochemicals which, as mentioned are toxic to biological growth. Therefore, commercially viable titers cannot be reached in the current respective fermentation approaches which rely on lignocellulosic biomass as the main carbon source.

Various lignocellulose-derived toxins, are generated during pretreatment processing of lignocellulosic material including furan derivatives (e.g. furfural (s); i5-hydromethylfurfural (HMF)), organic acids (e.g. acetic acid, formic acid, and ferulic acid) and lignin derivatives (e.g. vanillin, 4-hydroxybenzaldehyde, guaiacol, and phenol) (Palmqvist, Bioresource Technology 74, 2000, 17-24; Klinke, Appl Microbiol Biotechnol 66, 2004, 10-26). As mentioned, these toxins severely inhibit fermentation processes, for example the consequent enzymatic hydrolysis and ethanol fermentation (Jorgensen, Biofuels Bioprod Bioref 1, 2007, 119-134; Jing, Appl Biochem Biotechnol 159, 2009, 696-707).

Several strategies in research and industry have been reported to mitigate the deleterious effects of lignocellulose-derived toxins and to enable sufficient biological growth on lignocellulosic feedstocks. These strategies can be categorized as follows.

First, strategies to lower the toxin concentrations in lignocellulosic material-derived carbon sources (e.g. in lignocellulose hydrolysates) prior to the use of the same in subsequent fermentation processes. These strategies are also known as detoxification strategies. In view of the strong impact of lignocellulose-derived toxins on fermentation performance, a detoxification step to remove the toxins is often considered unavoidable for profitable fermentation. Detoxification is either tried through purification of lignocellulosic hydrolysates or through specific ways of producing the lignocellulosic hydrolysates. Removal of toxins from the hydrolysates was proposed by using physio-chemical or biological methods, as described in, for example, EP2342348B1, WO2010/037780, Liu (Gene 446, 2009, 1-10), Palmqvist (loc. cit.), Zhang (Biotechnol Biofuels 3 (26), 2010, 1-15), US-A1-2016/0002359 and WO2013/122917.

Second, strategies relying on genetic modification of the used microorganisms to improve their tolerance/resistance to lignocellulose-derived toxins and to improve their ability to grow on lignocellulosic material, respectively. Such genetic modification approaches are, for example described in Niehus (Biotechnol Biofuels 11(11), 2018, 1-10), Liu (loc. cit.), U.S. Pat. No. 8,936,929 and WO2011/079388.

The above strategies, however, incur significant drawbacks, like additional costs for the overall bioprocess development. These drawbacks render such processes commercially unattractive (e.g. unviable for commodity products). In addition, there are negative impacts on the target product or productivity. Further, the use of genetically modified organisms would render the resulting product incompatible with bio-organic labelling requirements and may, in consequence, affect consumer acceptance.

A further strategy has been reported to mitigate the deleterious effects of lignocellulose-derived toxins, namely the use of increased inoculums for the (production) fermentation. For example, Yu (Process Biochemistry 49, 2014, 457-465) attempted to partially overcome the toxic effect of lignocellulosic feedstocks by using a higher amount of inoculum (e.g. 10%). However, this process achieves at best 50% of the performance in normal media. Further, the benefit from inoculum increasement is not linear (e.g. a steep increase of the inoculum does not correlate to a steep increase of productivity; for example, a use of 20% inoculum does not lead to further improvement at all). This indicates that, even if the inoculum is increased, the lignocellulosic medium still exerts its toxic effect on the fermentation performance. An additional downside of this strategy is the need of higher fermentation device volumes to accommodate the high inoculum volumes.

There is thus still an unmet need in the field of microbial fatty acid/lipid/oil production when attempting to perform commercially attractive fermentation approaches on the basis of lignocellulose-derived carbon sources.

The problem underlying the present invention is therefore the provision of improved means and methods for a viable fermentation and production of oleaginous yeasts and/or fatty acids, lipids and oils using lignocellulosic material as the major carbon source (e.g. without the need of a costly removal of the toxic compounds and/or without the need to genetically modify the yeast strains to make them more resistant).

The technical problem is solved by the provision of the embodiments characterized in the claims.

The present invention relates to a method of producing an oleaginous yeast strain adapted to a lignocellulose medium, said method comprises, as the two main steps, the steps of
(i) growing oleaginous yeast cells in a medium which is essentially free of compounds which inhibit the growth of said oleaginous yeast cells and which are derivable from lignocellulosic material (“inhibitors” herein elsewhere), wherein said medium comprises at least one carbon source which is fermentable by said yeast cells (this step is also termed “first main step” herein); and
(ii) adding to said medium comprising said yeast cells a lignocellulosic preparation (including at least one of said inhibitors), and further growing said oleaginous yeast cells in the medium (this step is also termed “second main step” herein).

The method of the invention may comprise an additional, third step, namely the step of
(ii′) inducing/increasing lipogenesis in said oleaginous yeast cells (this step is also termed (optional) “third main step” herein).

The method of the invention may comprise an additional, fourth step, namely the step of
(iii) harvesting said oleaginous yeast cells (this step is also termed “fourth main step” herein).

It is preferred that this additional, fourth, main step is performed after step (ii), or after step (ii′), supra.

A three-step method in accordance with the invention is a method of producing an oleaginous yeast strain adapted to a lignocellulose medium, said method comprises, as the three main steps, the steps of
(i) growing oleaginous yeast cells in a medium which is essentially free of compounds which inhibit the growth of said oleaginous yeast cells and which are derivable from lignocellulosic material (“inhibitors” herein elsewhere), wherein said medium comprises at least one carbon source which is fermentable by said yeast cells;
(ii) adding to said medium comprising said yeast cells a lignocellulosic preparation (including at least one of said inhibitors), and further growing said oleaginous yeast cells in the medium; and
(ii′) inducing/increasing lipogenesis in said oleaginous yeast cells.

A four-step method in accordance with the invention is a method of producing an oleaginous yeast strain adapted to a lignocellulose medium, said method comprises, as the three main steps, the steps of
(i) growing oleaginous yeast cells in a medium which is essentially free of compounds which inhibit the growth of said oleaginous yeast cells and which are derivable from lignocellulosic material (“inhibitors” herein elsewhere), wherein said medium comprises at least one carbon source which is fermentable by said yeast cells;
(ii) adding to said medium comprising said yeast cells a lignocellulosic preparation (including at least one of said inhibitors), and further growing said oleaginous yeast cells in the medium;
(ii′) inducing/increasing lipogenesis in said oleaginous yeast cells; and
(iii) harvesting said oleaginous yeast cells.

In accordance with the invention, it is crucial that step (i) is performed before step (ii), and step (ii) is performed after step (i), respectively. It is preferred that optional step (ii′) is performed after step (ii) or during/as the terminal phase at the end of step (ii).

It is most preferred that the above steps are performed in this chronological order: step (i) (i.e. first step), step (ii) (i.e. second step), step (ii′) (i.e. optional third step), step (iii) (i.e. fourth step). (An) additional further (secondary) step(s) may be performed prior, after and/or in between these (main) steps

The first two main steps in accordance with the method of the invention (steps (i) and (ii), respectively) may also be described as follows (in an illustrative, non-limiting manner; further descriptions are given herein elsewhere):
First main step (growth stage; step (i)): In this (initial) stage, the biomass (oleaginous yeast cells) is grown in medium with (essentially) pure (non-lignocellulose derived) glucose as the fermentable carbon source (no inhibitor(s)) (or medium with (a) comparable non-lignocellulose derived sugar source(s) as replacer(s) or supplement(s) for the glucose), preferably under ample nutrition. This allows establishing a viable and healthy community of microorganisms (oleaginous yeast cells). This strategy of starting the culture/fermentation with (essentially) pure (non-lignocellulose derived) glucose (and/or with (a) comparable non-lignocellulose derived sugar source(s)) avoids, for example, the issues arising due to the inhibitors being front-loaded had the culture/fermentation been started with the lignocellulosic material/lignocellulosic preparation (e.g. growth inhibition).
Second main step (transition and adaptation stage; step (ii)): In this next stage, the oleaginous biomass and the (initial) medium which contains the same, respectively, is slowly transitioned from the (essentially) pure glucose (and/or comparable non-lignocellulose derived sugar source(s)) to the lignocellulosic material/preparation (typically including the inhibitor(s)). Without being bound by theory, the slow transition phase has a twofold effect: (i) it allows the microorganisms (oleaginous yeast cells) the time to undergo metabolic changes necessary to adapt to the lignocellulosic material/preparation, for example to resist the inhibitors present in the lignocellulosic material/preparation; and (ii) it acts as a (pseudo natural) selection within the localized microbial community (oleaginous yeast cells) and only allows the most robust cells to propagate and grow further (e.g. in the reactor). In the context of the invention, this adaptation has been shown by transcriptomic sequencing data. For example, metabolic stress response RPOS was shown to be activated.

The (optional) further third and fourth main steps in accordance with the methods of the invention (steps (ii′) and (iii), respectively) may also be described as follows (in an illustrative, non-limiting manner; further descriptions are given herein elsewhere):
Third main step (lipid accumulation stage; step (ii′)): In this stage, once the cell community has been (fully) transitioned to the lignocellulosic material/preparation, and stable growth parameters have been established, lipogenesis may be induced/increased. This may be done by removal/decrease of the nitrogen feeding regimen. Upon exhaustion of nitrogen (for example in the bioreactor), the microorganisms do no longer divide and propagate further and, instead, the excess carbon from the lignocellulosic material/preparation-derived carbon source (e.g. glucose) is channeled towards fatty acid/lipid/oil production and storage.
Fourth main step (step of harvesting the produced microorganisms; step (iii)): In accordance with this step, the microorganisms (oleaginous yeast cells), as grown/processed according to the (preceding) first and second (and optional third) steps are harvested (from the medium/fermenter/bioreactor).

The so-grown/processed (and harvested) oleaginous yeast cells constitute the oleaginous yeast strain or oleaginous yeast cells which is/are adapted to a lignocellulose medium in accordance with the invention.

The present invention solves the technical problem because, as documented herein below and in the appended examples, a particular two-stage, optionally three-stage or four-stage, oleaginous yeast fermentation process is provided which utilizes lignocellulosic material as the main carbon source (in particular a lignocellulose hydrolysate), and which results in yeast cell biomass and/or fatty acid/lipid/oil productivities which are comparable to productivities as achieved by non-lignocellulosic carbon source (e.g. glucose) utilization (e.g. without the need for removal of the inhibitor(s) and/or of genetic modifications; and without substantial negative effects on productivity).

In particular, it has surprisingly been found in the context of the invention that, once oleaginous yeast strains or oleaginous yeast cells are produced according to the methods of the invention, the yeast strains/cells adapt to the conditions occurring in lignocellulose media and resulting from added lignocellulosic preparation(s), in particular the toxic conditions. Without being bound by theory, the adaptation potentially comes along with no substantial changes in the DNA sequences, but with changes of the RNA expression profile. The adaptation may be evident on the transcriptomic level. The adaptation may result from changes in the mRNA expression profile; for example, transient changes in the mRNA expression of one or more (important) yeast stress marker(s) (e.g. one or more of the stress marker(s) as described herein below and/or in Example 7; e.g. key markers of MAPK stress response superfamily and zinc finger family oxidoreductases). The changes of the RNA expression profile could readily be analyzed by the skilled person and/or the adaptation could readily be verified by the skilled person (e.g. as in the appended examples). The potentially resulting RNA expression profile is expected to be characteristic for lignocellulose-adapted oleaginous yeast cells/strains according to the invention (i.e. adapted to the lignocellulose carbon source/medium/preparation according to the method(s)/method step(s) of the invention). Thus, adapted oleaginous yeast cells/strains are also provided, and can also be provided, in accordance with the invention. These may be stored (e.g. as (a) frozen stock(s)) and/or used in (a) continuous/separate fermentation process(es) (e.g. without the need to undergo the first and/or second main step(s) (growth stage and/or transition and adaptation stage) again/de novo).

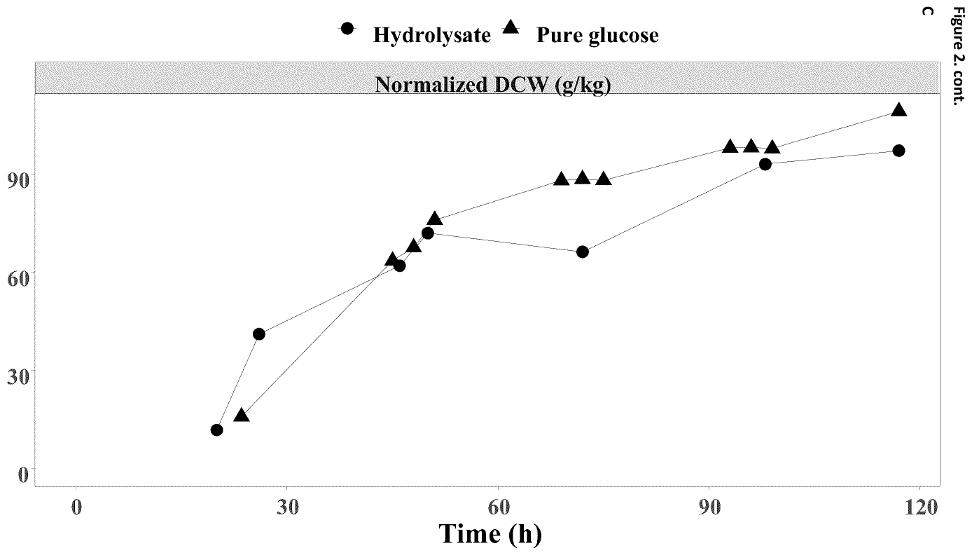

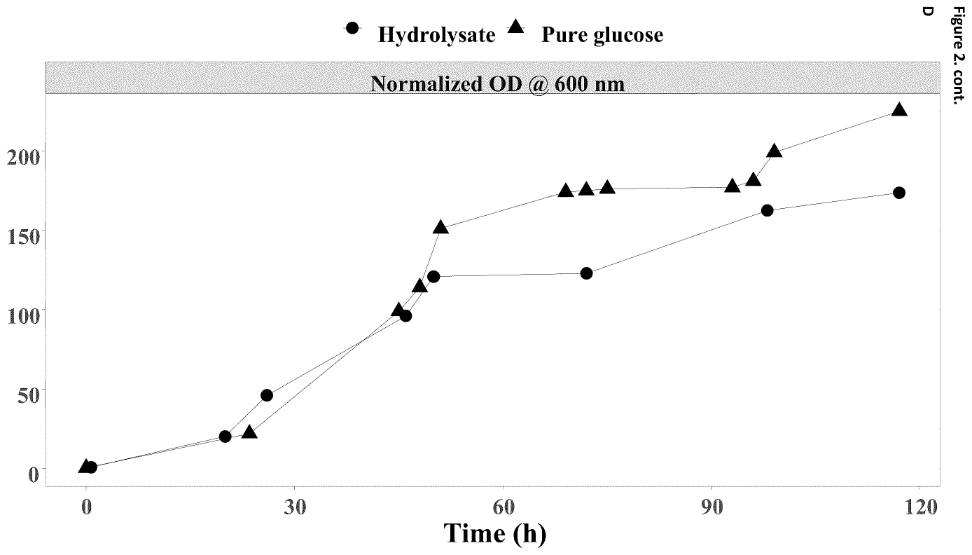

In addition, it is documented herein below and in the appended examples that, surprisingly, the oleaginous yeast cells/strains which are produced according to the methods of the invention show a fermentation performance (biomass and lipid production etc.) which equals the fermentation performance based on glucose (non-lignocellulose-derived) as the sole carbon source (see, for example, Examples 3, 4 and 5 and FIGS. 2, 3 and 4). In particular, it has been shown in the context of the present invention and the appended examples that, during main step (ii), and in the adapted oleaginous yeast strains/cells, neither carbon and nitrogen uptake nor cell growth was substantially impacted by the presence of lignocellulose-derived inhibitors. Further, oleaginous yeast cells/strains which have been adapted to lignocellulose medium in accordance with the invention show similar levels of lipid accumulation and lipid titers between pure glucose (non-lignocellulose-derived) and lignocellulose-derived carbon sources (mainly glucose (e.g. about 70%) and xylose (e.g. about 30%)); e.g. a lipid accumulation of about 50-70% (w/w). More particular, the final oleaginous yeast cell/strain growth and/or biomass and/or lipid production levels as achieved according to the methods of the invention equals the final oleaginous yeast cell/strain growth and/or biomass and/or lipid production levels, respectively, as achieved by non-lignocellulose-derived glucose.

One particular advantage of the invention is that, by applying the methods of the invention (in particular the first and second main steps), oleaginous yeast cells/strains can be adapted to a wide variety of different lignocellulose media, irrespective of the particular compositions of carbon source(es) and inhibitor(s) these may have. Once oleaginous yeast cells/an oleaginous yeast strain is first grown in a medium according to step (i) (essentially no inhibitor(s); preferably pure (non-lignocellulose derived) glucose (or (a) comparable non-lignocellulose derived sugar source(s) as replacer(s) or supplement(s) for the glucose); preferably ample nutrition), and the medium including the so grown cells/strain is then slowly transitioned into a lignocellulose medium (including the inhibitor(s)) by adding the lignocellulosic preparation according to step (ii), the cells/strain adapts to the lignocellulose medium so achieved, irrespective of the actual composition of the medium (e.g. kind and concentration of carbon source(s) and/or inhibitor(s)).

The meaning of “oleaginous yeast” is well known in the art and the term is accordingly used herein (cf., e.g., López, Yeast 39(11-12), 2022, 553-606). Typically, an “oleaginous yeast” in accordance with the invention is a yeast which cells are capable of producing/accumulating about ≥20% of their dry cell weight (dcw) as fatty acids, lipids and/or oils, in particular lipids and/or triacylglycerides. Preferred oleaginous yeasts are yeasts which cells are capable of producing/accumulating about ≥30%, about ≥40% and even about ≥50% of their dcw as fatty acids, lipids and/or oils, in particular lipids and/or triacylglycerides (for example about 50%-70% of their dcw).

Particular, non-limiting examples of oleaginous yeasts which may be used in accordance with the invention are yeasts of the genus (and species) selected from the group consisting of Rhodotorula (e.g. R. toruloides, R. babjevae, R. diobovata, R. glutinis, R. mucilaginosa, R. kratochvilovae), Yarrowia (e.g. Y. lipolytica), Cutaneotrichosporon (e.g. C. curvatus, C. guehoae), Naganisha (e.g. albida), Cryptococcus (e.g. C. ramirezgonezianus, C. terricola, C. curvatus), Solicoccozyma (e.g. S. phenolicus), Papiliotrema (e.g. P. baii), Myxozyma (e.g. M. mucilagina, M. melibiosi), Vishniacozyma (e.g. V. aff. heimaeyensis), Vanrija (e.g. V. musci), Leucosporidium (e.g. L. scottii), Scheffersomyces (e.g. S. stipitis), Lipomyces (e.g. L. starkeyi), Candida (e.g. C. insectorum), Filobasidium (e.g. F. magnum, F. wieringae), Rhodosporidium (e.g. R. fluvialis), Metschnikowia (e.g. M. pulcherrima), Wickerhamomyces (e.g. W. siamensis).

A “lignocellulose medium” in accordance with the invention is a medium which contains lignocellulosic material (also termed lignocellulose material, lignocellulosic/lignocellulose feedstock, lignocellulosic/lignocellulose biomass), in particular (a) lignocellulosic preparation(s) (also termed (a) lignocellulose preparation(s)). Lignocellulose media, and their contents, preparations etc., as well as lignocellulosic materials, and their origins, contents, preparations etc., are well known in the art (cf., e.g., EP2342348B1; WO2010/037780; Liu (loc. cit.); Palmqvist (loc. cit.); Zhang (loc. cit.); US-A1-2016/0002359; WO2013/122917; Niehus (loc. cit.); Liu (loc. cit.); U.S. Pat. No. 8,936,929; WO2011/079388). A non-limiting example of a “lignocellulose medium” is provided in Examples 1, 3 and 4 (cf. also Tables 5 and 6). A “lignocellulose medium” in accordance with the invention (typically) contains both, (i) at least one carbon source (or a mixture of at least two different carbon sources; e.g. glucose and xylose) which is/are derived from lignocellulosic material and which is/are fermentable by an oleaginous yeast/oleaginous yeast cells/an oleaginous strain; and (ii) at least one compound which is derived from lignocellulosic material and which is capable of inhibiting the growth of oleaginous yeast cells (inhibitor(s)). A “lignocellulose medium” in accordance with the invention is not limited to a particular lignocellulose medium with (a) particular kind(s) and concentration(s) of lignocellulose-derived carbon source(es) and/or (a) particular kind(s) and concentration(s) lignocellulose derived of (a) inhibitor(s). The skilled person knows typical lignocellulose media, their compositions, origins, production methods etc. (see, for example, the above cited references). Preferably, a lignocellulose medium in accordance with the invention contains an amount of a lignocellulosic preparation so that (a) kind(s) and (an) amount(s) of (a) carbon source(s) which is typical for media in oleaginous yeast fermentation occurs in the medium (e.g., for a batch fermentation about 60 g/kg medium (for example about 60 g glucose/kg medium or about 60 g glucose (e.g. 77%) and xylose (e.g. 22%)/kg medium)). The amount of a lignocellulosic preparation to be added to meet the need for the carbon source by the yeast cells may determine the amount(s) of inhibitor(s) which are introduced into the medium via the lignocellulosic preparation.

The meaning of “lignocellulosic material” is likewise well known in the art and the term is accordingly used herein (cf., e.g., Yu loc. cit.; Malherbe, Reviews in Environmental Science and Biotechnology 1, 2002, 105-14). Typically, lignocellulosic material is, is part of, or is derived from organic waste (mostly plant material-derived waste), like straw, stalk, dung, bagasse, pomace, foliage, wood-derived waste, agriculture-derived waste, agricultural residues etc. Typical main constituents of lignocellulosic material are cellulose, hemicellulose and/or lignin (cf., e.g., Yu loc. cit.; Malherbe loc. cit.). Typically, cellulose is a linear polymer of glucose, hemicellulose is a heteropolysaccharide composed of abundant pentoses (xylose, arabinose, etc.) and a few hexoses (glucose, galactose, mannose, rhamnose, etc.), and lignin is an aromatic polymer composed of phenylpropane subunits (see, e.g., Yu loc. cit.; Almeida, Journal of Chemical Technology and Biotechnology 82, 2007, 340-9).

The meaning of “lignocellulosic preparation” is also well known in the art and the term is accordingly used herein. Typically, lignocellulosic preparations are “lignocellulosic hydrolysates” (also termed “lignocellulose hydrolysates”) (cf., e.g., EP2342348B1; WO2010/037780; Liu (loc. cit.); Palmqvist (loc. cit.); Zhang (loc. cit.); US-A1-2016/0002359; WO2013/122917; Niehus (loc. cit.); Liu (loc. cit.); U.S. Pat. No. 8,936,929; WO2011/079388; Malherbe loc. cit.). In particular, a lignocellulosic preparation (e.g. lignocellulosic hydrolysate) in accordance with the invention results from processing lignocellulosic material so that at least one carbon source (predominantly sugar) which is fermentable by oleaginous yeast cells/an oleaginous yeast/strain is obtained. Typically, a lignocellulosic preparation (e.g. lignocellulosic hydrolysate) in accordance with the invention is obtained by hydrolyzing cellulose and/or hemicellulose as contained in a lignocellulosic material. Prior to hydrolyzation, (a) pretreatment process(es) may be performed (for example disruption of cell walls of plant material as comprised in lignocellulosic material; see below for further details).

“Lignocellulosic material” and a “lignocellulosic preparation” may, for example, be derived from wood, bark, wheat plants, barley plants, rice plants, potato plants, sorghum plants, rye plants, oat plants, maize/corn plants, legume plants (e.g. soja plants), agave plants, sugar cane plants, other crop plants, flowers, foliage, and the like.

A “lignocellulosic preparation” (e.g. lignocellulosic hydrolysate), and “lignocellulose medium”, respectively, in accordance with the invention contains at least one carbon source (predominantly sugar(s)); or a mixture of at least two different carbon sources (e.g. glucose and xylose) which is derived from lignocellulosic material and which is fermentable by an oleaginous yeast/oleaginous yeast cells/an oleaginous strain. This means that, in principle, any lignocellulosic preparation which provides at least one carbon source which is fermentable by an oleaginous yeast may be used in accordance with the invention (in particular in the context of the second main step). In particular, a lignocellulosic preparation (e.g. lignocellulosic hydrolysate), and lignocellulose medium, respectively, in accordance with the invention may contain at least one lignocellulosic material-derived fermentable carbon source selected from the group consisting of glucose (dextrose), xylose, arabinose, galactose, mannose, rhamnose, etc. Xylose is a preferred, glucose (dextrose) is the most preferred lignocellulosic material-derived carbon source in the lignocellulosic preparation (e.g. lignocellulosic hydrolysate), and lignocellulose medium, respectively. A mixture of at least glucose and xylose may typically be contained as fermentable carbon sources in the “lignocellulosic preparation” (e.g. lignocellulosic hydrolysate), and “lignocellulose medium”, respectively. For example, a “lignocellulosic preparation” (e.g. lignocellulosic hydrolysate) may contain about 5% to 60% w/w glucose, 10% to 50% w/w glucose or 20% to 40% w/w glucose (for example about 30% w/w glucose) and(/or) about 1% to 30% w/w xylose, 4% to 20% w/w xylose, 5% to 10% w/w xylose (for example about 7% w/w xylose). “Lignocellulose medium” (for example, when used in batch fermentation) may contain glucose and xylose at comparable ratios. The amount of the (overall) carbon source(s) in the lignocellulose medium (comprising for example glucose and/or xylose) (for example when used in batch fermentation) may be diluted as compared to the lignocellulosic preparation as such (see, for example, above) by a factor of about 5 to 30, 10 to 25 or 16 to 18 (e.g. about 17). A lignocellulose medium (for example, when used in batch fermentation) may comprise about 0.5% to 30%, 1% to 20%, 3% to 10% or 4% to 8% (w/w) lignocellulosic preparation (e.g. 6%). In, for example, feeding phases (for example in the context of fed-batch fermentation), or in continuous fermentation, such amounts of lignocellulosic preparation in lignocellulose medium may only be contained after (a calculated) accumulation during or at the end of the feeding phase due to the (e.g. continuous) feeding (the absolute concentration(s)/amount(s) of fermentable carbon source(s) in the medium may be low, due to the (continuous) consumption by the yeast cells).

It is preferred that the fermentable carbon source(s) as contained in the “lignocellulosic preparation” (e.g. lignocellulosic hydrolysate) to be used in step (ii), and in the “lignocellulose medium”, respectively, is (are) predominantly derived from lignocellulosic material. However, small/some amounts of (a) non-lignocellulosic material-derived carbon source(s) may also be contained. It is envisaged that at least about 75%, 80%, 85%, 90%, 95%, 98% or 99% of the fermentable carbon source(s) (e.g. glucose (dextrose), xylose and/or arabinose etc.) as contained in the “lignocellulosic preparation” (e.g. lignocellulosic hydrolysate), and “lignocellulose medium”, respectively, to be used in accordance with the invention are derived from lignocellulosic material. Preferably about 100% of the fermentable carbon source(s) (e.g. glucose (dextrose), xylose and/or arabinose etc.) as contained in the “lignocellulosic preparation” (e.g. lignocellulosic hydrolysate), and “lignocellulose medium”, respectively, to be used in accordance with the invention are derived from lignocellulosic material.

Vice versa, it is envisaged that no more than about 25%, 20%, 15%, 10%, 5%, 2% or 1% of the fermentable carbon source(s) (e.g. non-lignocellulose-derived glucose (dextrose), isoglucose and/or DE95; or a mixture of two or more of glucose/dextrose, isoglucose and/or DE95 etc.) as contained in the “lignocellulosic preparation” (e.g. lignocellulosic hydrolysate), and “lignocellulose medium”, respectively, to be used in accordance with the invention are non-lignocellulosic material-derived. Preferably about 0% of the fermentable carbon source(s) (e.g. non-lignocellulose-derived glucose (dextrose), isoglucose, DE95 etc. as disclosed herein) as contained in the “lignocellulosic preparation” (e.g. lignocellulosic hydrolysate), and “lignocellulose medium”, respectively, to be used in accordance with the invention are non-lignocellulosic material-derived.

A “lignocellulosic preparation” (e.g. lignocellulosic hydrolysate), and “lignocellulose medium”, respectively, in accordance with the invention typically further contains (besides the carbon source(s)) one or more compound(s) which (i) inhibit(s), or is/are capable of inhibiting, the growth of oleaginous yeasts cells (in particular of oleaginous yeasts cells which are not adapted to a lignocellulose medium in accordance with the invention), and which (ii) is/are derivable from lignocellulosic material. This/these compound(s) is/are also termed “inhibitor(s)” herein. Such inhibitor(s) is/are typically present in the medium (specifically in step (ii)) in (an) amount(s) sufficient to inhibit(s) the growth of oleaginous yeasts cells (non-adapted). Such inhibitors usually emerge upon the processing of lignocellulosic materials into lignocellulosic preparations (see above and, e.g., Palmqvist loc. cit.; Klinke loc. cit.). For example, such inhibitors emerge upon pretreatment processes (e.g. prior to hydrolysation), like pretreatment processes which are required to disrupt cell walls of plant material as comprised in lignocellulosic materials (e.g. acid or stream pretreatments). In principle, a “lignocellulosic preparation” (e.g. lignocellulosic hydrolysate), and “lignocellulose medium”, respectively, in accordance with the invention may contain (mixtures of) (a) fermentable carbon source(s) but no inhibitor(s). Such a “lignocellulosic preparation” (e.g. lignocellulosic hydrolysate), and “lignocellulose medium”, respectively, however, is untypical. In the context of one aspect of the invention, such an untypical “lignocellulosic preparation” (e.g. lignocellulosic hydrolysate), and “lignocellulose medium”, respectively, is excluded.

Lignocellulosic material-derived inhibitors of oleaginous yeast cell growth are well known in the art (see, e.g., Niehus loc. cit.; Yu loc. cit.; Yang, Front. Bioeng. Biotechnol. 6, 2018, 1-14 (https://www.frontiersin.org/articles/10.3389/fbioe.2018.00023). Typically, inhibitors of oleaginous yeast cell growth are divided in to three main groups: weak acids, furan derivatives, phenolic compounds. Inhibitors may include furan derivatives (like, e.g., furfural or 5-hydromethylfurfural (HMF)), organic acids (like, e.g. acetic acid, formic acid, ferulic acid) and lignin derivatives (like, e.g. vanillin, 4-hydroxybenzaldehyde, guaiacol, phenol). Examples of such inhibitors are also listed in Table 7, below. Particular inhibitors may be selected from the group consisting of:
(i) furfural(s);
(ii) 5-hydroxymethyl-furfural (HMF);
(iii) acetic acid;
(iv) formic acid;
(v) levulinic acid; and
(vi) syringaldehyde;
(vii) p-hydroxybenzaldehyde (PHB); and
(viii) vanillin.

An example of a typical (however, non-limiting) “lignocellulosic preparation” (e.g. lignocellulosic hydrolysate), and “lignocellulose medium”, respectively, in accordance with the invention contains at least one furfural (e.g. HMF).

A “lignocellulosic preparation” (e.g. lignocellulosic hydrolysate), and “lignocellulose medium”, respectively, in accordance with the invention may contain one or more inhibitor(s), for example (at least) 2, 3, 4, 5, 6 or 7 inhibitors (at least one, some or all at growth-limiting concentrations in relation to the medium; see below for further details).

Typical lignocellulosic preparations (containing the at least one fermentable carbon source and the at least one inhibitor), e.g. lignocellulosic hydrolysates, can be produced by various methods and by multiple manufacturers (e.g. Clariant, ADM, Cargill, Evonik, Fibers 365). An overview over lignocellulosic hydrolysates is given in Dahmen (GCB Bioenergy 11, 2019, 107-17; https://onlinelibrary.wiley.com/doi/pdf/10.1111/gcbb.12586). Various methods for the preparation of lignocellulosic hydrolysates are provided by, for example, section 4 of Dahmen (loc. cit.). Most relevantly, lignocellulosic hydrolysates can be produced by (chemically and/or enzymatically) hydrolyzing cellulose and/or hemicellulose as contained in/derived from a lignocellulosic material (for example according to the patent application MX/E/2015/084126). Single-step (e.g. chemical) treatment processes or two-step (e.g. chemical pretreatment and/or enzymatic hydrolysis) treatment processes may be applied in this respect. Prior to hydrolysation, pretreatment processes may be performed (e.g. (hydrochloric) acid pretreatment processes or steam pretreatment processes; cf., e.g., Palmqvist loc. cit.), in particular pretreatment processes which disrupt/degrade cell walls of plant material as comprised in lignocellulosic materials.

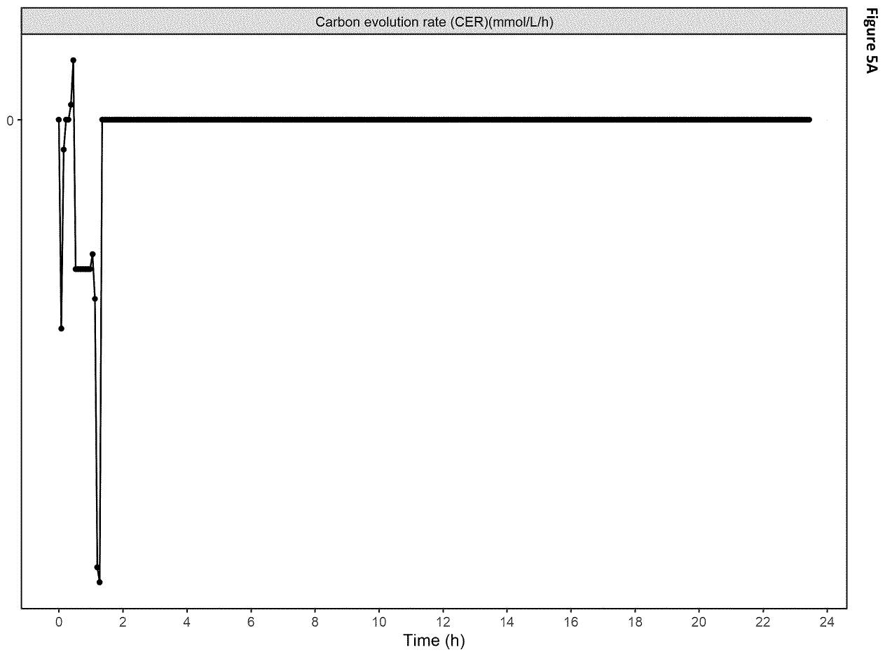

It is envisaged in the context of the invention that the “lignocellulose medium” (and medium in step (ii) (second main step) of the inventive method, at least at one timepoint) contains one or more inhibitor(s) at (a) concentration(s) so that the growth of oleaginous yeast cells (non-adapted in accordance with the invention) is inhibited (as compared to a comparable medium without, or substantially free of, the inhibitor(s); for example as compared to a medium as in step (i) (first main step described herein). Growth inhibition in this respect may be growth inhibition by at least about 5%, 10%, 20%, 30%, 40%, 50%, 60%, 70%, 80%, at least 90% or at least 95% (as, for example, determined on the basis of the OD₆₀₀, the wet cell weight (wcw) and/or the dry cell weight (dcw) of the oleaginous yeast cells in said medium (w/w); see the appended examples, Niehus (loc. cit.) and Yu (loc. cit.) for respective arrays). Even growth inhibition by up to 100% (±1-2%) is possible (cf. Example 6/FIG. 5).

The person skilled in the art is readily able to determine whether a lignocellulose medium (and a medium in step (ii) (second main step) of the inventive method, at least at one timepoint) contains one or more inhibitor(s) at (a) concentration(s) so that the growth of oleaginous yeast cells (non-adapted in accordance with the invention) is indeed inhibited. Respective means and methods are well known in the art (see, e.g. Yang loc. cit.; Niehus loc. cit.; Yu loc. cit.) and described in the appended examples. Typically, in lignocellulose media, the concentration(s)/amount(s) of inhibitors is/are at ppm to g/L levels.

A non-limiting selection of (a) particular inhibitor(s) and respective concentration(s) which may be contained in the lignocellulose medium (or (at one timepoint) in the medium in step (ii) (second main step) of the inventive methods (preferably at the end thereof)) is given in the following:
(i) furfural(s); at a concentration of, for example, about 0.01 g/l to 10 g/l, 0.1 g/l to 5 g/l or 0.3 g/l to 3.5 g/l (e.g. about 0.3 g/l, 0.6 g/l, 1 g/l, 1.5 g/l, 2 g/l, 2.5 g/l, 3 g/l, 3.5 g/l);
(ii) 5-hydroxymethyl-furfural (HMF); at a concentration of, for example, about 1 mM to 150 mM, 5 mM to 100 mM or 10 mM to 60 mM (e.g. about 5 mM, 10 mM, 20 mM, 30 mM, 40 mM, 50 mM, 60 mM, 70 mM, 80 mM, 90 mM);
(iii) acetic acid; at a concentration of, for example, about 0.01 g/l to 10 g/l, 0.1 g/l to 5 g/l or 0.3 g/l to 3.5 g/l (e.g. about 0.3 g/l, 0.6 g/l, 1 g/l, 1.5 g/l, 2 g/l, 2.5 g/l, 3 g/l, 3.5 g/l);
(iv) formic acid; at a concentration of, for example, about 0.002 g/l to 2 g/l, 0.02 g/l to 1 g/l or 0.05 g/l to 0.6 g/l (e.g. about 0.05 g/l, 0.1 g/l, 0.2 g/l, 0.3 g/l, 0.4 g/l, 0.5 g/l, 0.6 g/l, 0.7 g/l);
(v) levulinic acid; at a concentration of, for example, about 0.002 g/l to 2 g/l, 0.02 g/l to 1 g/l or 0.05 g/l to 0.6 g/l (e.g. about 0.05 g/l, 0.1 g/l, 0.2 g/l, 0.3 g/l, 0.4 g/l, 0.5 g/l, 0.6 g/l, 0.7 g/l);
(vi) syringaldehyde; at a concentration of, for example, about 0.1 g/l to 5 g/l, 0.3 g/l to 3 g/l or 0.5 g/l to 2 g/l (e.g. about 0.1 g/l, 0.3 g/l, 0.5 g/l, 1 g/l, 1.5 g/l, 2 g/l, 2.5 g/l, 3/l, 4 g/l, 5 g/l);
(vii) p-hydroxybenzaldehyde (PHB); at a concentration of, for example, about 0.05 g/l to 2.5 g/l, 0.15 g/l to 1.5 g/l or 0.25 g/l to 1 g/l (e.g. about 0.05 g/l, 0.15 g/l, 0.25 g/l, 0.5 g/l, 0.75 g/l, 1 g/l, 1.25 g/l, 1.5/1, 2 g/l, 2.5 g/l); and/or
(viii) vanillin; at a concentration of, for example, about 0.01 g/l to 5 g/l, 0.1 g/l to 5 g/l, 0.3 g/l to 3 g/l or 0.5 g/l to 2 g/l (e.g. about 0.01 g/l, 0.1 g/l, 0.3 g/l, 0.5 g/l, 1 g/l, 1.5 g/l, 2 g/l, 2.5 g/l, 3/l, 4 g/l, 5 g/l).

The respective kind(s) and/or concentration(s) of the inhibitor(s) which may be contained in the lignocellulosic preparation as such (for example to be added to the medium according to the (second main step) of the inventive methods; or to be used for preparing a lignocellulosic medium) may, in principle, be similar as the kind(s)/concentration(s) given for the inhibitor(s) in the lignocellulose medium (or in the medium in step (ii)). However, since the lignocellulosic preparation typically is diluted when added to a medium so as to achieve a lignocellulose medium (or upon addition to the medium according to step (ii)), concentration(s) of the inhibitor(s) which may be contained in the lignocellulosic preparation as such is/are usually higher than the concentration(s) given for the inhibitor(s) in the respective lignocellulose medium (or in the medium in step (ii)); e.g. by a factor of at least about 2, 3, 4, 5, 6, 7, 8 or 10, preferably by a factor of at least about 3, 4, 5, 6 or 7. Non limiting examples of concentrations of inhibitors in the lignocellulosic preparation as such are concentrations of, for example, about 0.1 g/l to 10 g/l, 0.5 g/l to 5 g/1, 1 g/l to 4.5 g/l or 2.5 g/l to 4 g/l (e.g. for furfurals or acetic acid), or about 0.1 g/l to 1.2 g/1, 0.2 g/l to 1 g/1, 0.4 g/l to 0.8 g/l or 0.5 g/l to 0.7 g/l (e.g. for formic acid).

In principle, a lignocellulosic preparation in accordance with the invention contains one or more inhibitor(s) at (a) concentration(s) so that, once the lignocellulosic preparation is contained in/added to a/the lignocellulose medium (or the medium in step (ii) (second main step)) of the inventive methods, the growth of oleaginous yeast cells (non-adapted in accordance with the invention) would indeed be inhibited in the lignocellulose medium (or the medium in step (ii)). What has been said with respect to growth inhibition/growth limitation herein above/elsewhere, applies here, mutatis mutandis.

A non-limiting selection of some potential inhibitors, and potential respective concentrations, in a lignocellulosic preparation, is also given in Niehus (loc. cit.) and in Table 7, below.

In any case, typical lignocellulose media (containing typical lignocellulosic preparations) contain both, (a) fermentable carbon source(s) and growth-inhibiting kind(s) and concentration(s) of inhibitor(s). In continuous mode fermentations, for example, the apparent carbon source(s) concentration in the medium may be low or even zero (e.g. ≤0.5% (w/w); the continuous feed may accord the need of the yeast cells). However due to the (continuous) feeding, the inhibitor(s) reach a (calculated) level in the medium which is growth-limiting (at least at one time point).

In principle, any carbon source(s) fermentable by oleaginous yeast cells, at any suitable concentration(s), may be contained in the medium in step (i) (first main step). Suitable carbon sources and suitable concentrations can readily be chosen by the skilled person. Respective non-limiting examples are disclosed herein and in the appended examples (e.g. Examples 1, 3 and 4), Table 3 as well as in Niehus (loc. cit.) and Yu (loc. cit.). Particular carbon sources which may be contained in the medium in step (i) are selected from the group consisting of (pure) glucose/dextrose (preferred), isoglucose, xylose, fructose, sucrose and DE95. The use of a mixture of two or more of glucose/dextrose, isoglucose, xylose, fructose, sucrose and DE95 is also envisaged herein. The medium in step (i) may comprise, per kg of medium, between from about 10 g to about 200 g or, preferably, between from about 30 g to about 120 g of (a) fermentable carbon source(s) (e.g. about 40 g, 60 g or 80 g; of, for example, (pure) glucose/dextrose (preferred); supplements or alternatives (alone or as mixtures) are isoglucose, xylose, fructose, sucrose, DE95).

The kind and/or concentration/amount of the fermentable carbon source(s) contained in the medium in step (i) will be chosen by the skilled person so that the oleaginous yeast cells are allowed to grow well and establish a viable/healthy community and/or that the needs of the fermentation mode is met (e.g. both, fed-back, continuous), etc.

It is preferred that the medium in step (i) does not comprise a carbon source which is derived from lignocellulosic material and which is provided by a lignocellulosic preparation, respectively. However, the at least one carbon source which is fermentable by the yeast cells may also be lignocellulose-derived, provided that no (essential amounts of) compounds which inhibit the growth of the oleaginous yeast cells would be present in the medium in step (i). For example, the medium in step (i) may comprise ((a) carbon source(s) provided by) a detoxified lignocellulosic preparation (for example as disclosed in the context of EP2342348B1, WO2010/037780, Liu (loc. cit.), Palmqvist (loc. cit.), Zhang (loc. cit.), US-A1-2016/0002359 and WO2013/122917), provided that no (essential amounts of) compounds which substantially inhibit the growth of the oleaginous yeast cells is provided to the medium in step (i) by the detoxified lignocellulosic preparation.

In any case, the medium in step (i) is (essentially) free of compounds which inhibit the growth of said oleaginous yeast cells and which are derivable from lignocellulosic material (also “inhibitors” herein elsewhere). It is most preferred that no lignocellulosic preparation at all, and no lignocellulosic preparation-derived carbon source, is contained the medium in step (i). “Essentially free” in this respect means that no (substantial amounts of) compounds which inhibit the growth of the oleaginous yeast cells are present in the medium in step (i) (for example at or, preferably, below ppb levels; most preferably zero). Minor (non-inhibiting) amounts of inhibitors and/or some minor growth inhibition in the medium in, and during, step (i), however, is also within the scope of the invention (for example amounts of inhibitors below the (ranges of) inhibitor concentrations given herein elsewhere for the lignocellulosic medium and/or a growth inhibition of at most 10%, 5%, 3% or, preferably, 1% (as compared to a comparable medium entirely without the inhibitor(s)).

In principle, any suitable duration/period for step (i) (“period” in the following) may be applied in the context of the invention (e.g. starting with the initial inoculum; EFT). The skilled person is readily able to choose respective suitable periods, for example for a given fermentation setting. In particular, the skilled person is readily able to choose the period of step (i) so that the oleaginous yeast cells are allowed to establish a viable/healthy community. Periods of typical batch fermentation settings, more particular of batch phases of typical fed-batch fermentation settings may, for example be applied. For example, the period of step (i) may be a period of about 10 to about 100 hours, about 15 to about 70 hours, about 20 to about 50 hours. Preferred periods are about 10 hours to about 50 hours, about 15 hours to about 40 hours or about 20 hours to about 35 hours (e.g. about 25 hours, 28 hours, 30 hours). In principle, also particularly short and long periods are envisaged (e.g. 15 hours, 10 hours or even less (e.g. 6 to 8 hours); and 100 hours, 150 hours or even more (e.g. 1, 2, 3, 4 or even more weeks)). These particularly long and short periods are, however, less preferred.

In case step (i) is conducted as a batch/batch phase, it is preferred that step (i) is conducted until most, or essentially all, of the (initial and/or (further) bolus-wise provided) fermentable carbon source has been consumed by the oleaginous yeast cells. For example, step (i) may be conducted until at least about 50%, 60%, 70%, 80%, 90%, 95% (preferred), or almost 100% (±1%-2%) of the fermentable carbon source has been consumed by the oleaginous yeast cells.

Generally, step (i) is to be conducted until a viable and/or healthy oleaginous yeast cell culture is established; for example, until the culture has reached (the start of) the exponential growth phase (for example after the initial inoculum). More particular, step (i) may be conducted until a substantial part of the (expected) final yeast cell biomass has been reached (the (expected) final yeast cell biomass at the end of step (ii), optionally including step (ii′)). For example, step (i) may be conducted until (at least) about 5% to 50%, about 10% to 45% or about 20% to 40% of the final (expected) yeast cell biomass has been reached in the medium (e.g. of about 10%, 15%, 20%, 25%, 30% or 35%). In this context, the yeast cell biomass may be determined on the basis of the OD₆₀₀, the wet cell weight (wcw) and/or the dry cell weight (dcw) of said yeast cells in said medium (w/w)). Further, step (i) may be conducted until an OD₆₀₀ of said yeast cells in said medium of about 20 to 100, of about 30 to 90, of about 40 to 80 or of about 50 to 70 has been reached (e.g. an OD₆₀₀ of about 60). Further, step (i) may be conducted until said yeast cells in said medium have reached a wcw of about 30 g to 300 g, of about 60 g to 240 g or of about 80 g to 160 g per kg medium (e.g. a wcw of about 120 g per kg medium). Further, step (i) may be conducted until said yeast cells in said medium have reached a dcw of about 10 g to 80 g or about 20 g to 50 g per kg medium (preferably about 30 g to 40 g per kg medium; e.g. about 35 g per kg medium). Further, step (i) may be conducted until the dissolved oxygen (pO₂) has reached a value of ≥50% (e.g. 50% to 100%) of DO saturation in the medium. Further, step (i) may be conducted until a drop in off-gas CO₂ signal to ≤1.5% occurs in the medium. Respective assays for determining the above parameters, and thus the endpoint of step (i), are known in the art (e.g. as cited in the above background reaction) and are also provided in the appended examples.

The yeast cells in step (i) may be grown under typical/appropriate nutrition conditions for oleaginous yeast cells. These conditions art know in the art (e.g. Niehus loc. cit.; Yu loc. cit.; López loc. cit.). Further, such conditions are exemplified in the appended examples (e.g. Examples 1, 3 and 4; Tables 3 and 4). It is preferred that the yeast cells in step (i) are grown under ample or even optional nutrition conditions, for example conditions with no nitrogen limitation (see, e.g. the above cited art and Examples 1, 3 and 4; Tables 3 and 4).

Further, suitable media, in particular basic media for (growth/fermentation of) oleaginous yeast cells are known in the art (see above) and are also described in the appended examples (see above). Besides the carbon source(s), a (basic) media for oleaginous yeast cells may comprise a yeast extract (or (an) comparable extract(s)), calcium sulfate (dihydrate), potassium sulfate, magnesium sulfate (heptahydrate), citric acid, potassium phosphate (monobasic), ammonium sulfate, zinc sulfate (heptahydrate), antifoam, copper sulfate (pentahydrate), manganese sulfate (monohydrate) and/or ferrous sulfate (heptahydrate). Sodium hydroxide, hydrochloric acid, ammonium hydroxide and/or acetic acid may also be comprised. Non-limiting examples of media to be applied in step (i) are described in Examples 1, 3 and 4 and by Tables 3 and 4. With the exception of the origin of the carbon source(s) and inhibitor(s) (and optionally the nitrogen availability), the (basic) media in step (i), in step (ii) (optionally including step (ii′)) and the lignocellulose medium may be similar or even the same.

By adding a lignocellulosic preparation according to step (ii) (second main step) to a medium comprising the grown yeast cells according to step (i), said medium including the yeast cells is slowly, i.e. non-abruptly, transitioned to a lignocellulose medium (in particular, the adding is envisaged to result in the presence of only (a) small (non-growth-limiting) amount(s) of inhibitor(s), in particular at the beginning/during first period of step (ii); e.g. at ppb levels). The adding of a lignocellulosic preparation so that a lignocellulose medium is abruptly formed (which would result in growth inhibition of the (non-adapted) yeast cells) is excluded from step (ii) (for example an adding which abruptly forms a lignocellulose medium which contains (a) growth-limiting kind(s)/amount(s) of inhibitor(s) for non-adapted yeasts; e.g. at ppm to g/l levels). In fact, the adding of a lignocellulosic preparation according to step (ii) is so that no abrupt, but a non-abrupt/slow, formation of a lignocellulose medium occurs. Some minor growth limitation (e.g. ≤10%, preferably ≤5%) may, however, be tolerated (in particular at the beginning of step (ii); not yet adapted yeast cells) Thus, at the beginning of step (ii), the medium does not constitute a lignocellulose medium (e.g. a medium with (a) concentration(s) of (the) inhibitor(s) which equal(s)/approximate(s) (the) concentration(s) of (the) inhibitor(s) of a lignocellulose medium, in particular of the lignocellulose medium to which the cells are to be adapted; e.g with (a) kind(s)/amount(s) of inhibitor(s) for non-adapted yeasts at ppm to g/l levels). The concentration(s) of (the) inhibitor(s) as occurring in a/the lignocellulose medium is reached slowly, i.e. non-abruptly, during the course of step (ii).

Adding a lignocellulosic preparation to a medium comprising the yeast cells as grown according to step (i) so that a lignocellulose medium is abruptly formed (for example in form of one single bolus) is not within the scope of the invention. Likewise, transferring (parts of) the medium comprising the yeast cells as grown according to step (i) and/or (parts of) the yeast cells as grown according to step (i), for example in form of an inoculum, to a medium which already contains a lignocellulosic preparation so that a lignocellulose medium including the yeast cells is abruptly formed upon the transfer is not within the scope of the invention. However, transferring, for example, (parts of) the medium comprising the yeast cells as grown according to step (i) and/or (parts of) the yeast cells as grown according to step (i) to a new medium (e.g. a new medium as in step (i)), and then adding a lignocellulosic preparation to the new medium containing the yeast cells according to step (ii), is withing the scope of the invention (slow, non-abrupt transition). In such case, the new medium may even contain some minor initial amount of lignocellulosic preparation (growth limitation ≤10%, preferably ≤5%, as long as it does not already constitute the full lignocellulose medium before the adding starts (i.e. no full concentration(s) of the inhibitor(s)).

The adding in accordance with the step (ii) invention allows the oleaginous yeast cells to adapt to the lignocellulose medium to be formed (for example by undergoing metabolic changes necessary to resist the inhibitor(s) present in the lignocellulosic preparation and resulting medium, respectively, and/or due to the (pseudo natural) selection within the oleaginous yeast cells). As a result of the adding in accordance with step (ii), these is no typical early-phase “depression” and/or typical reduction of the final yeast biomass as occurring once the yeast cells would abruptly be transferred to a lignocellulose medium (for example in form of an inoculum) or, in other words, once the inhibitor(s) would be front-loaded by starting already the growth stage with a lignocellulose medium). Differently spoken, in the context of the invention, the lignocellulosic preparation (containing the carbon source(s) fermentable by an oleaginous yeast and the inhibitor(s)) is added according to step (ii) to the medium comprising the yeast cells as resulting from step (i) so that the oleaginous yeast cells adapt to the lignocellulosic preparation-containing medium in accordance with the invention. In particular, the adding is in accordance with the herein described slow transition from the (essentially) pure non-lignocellulose derived carbon source(s) (step (i)) to the lignocellulosic preparation and medium containing the same (the medium (at the end) of step (ii)). That is, the lignocellulosic preparation is added according to step (ii) so that the oleaginous yeast cells are able to undergo metabolic changes necessary to resist the inhibitor(s) present in the lignocellulosic preparation; and/or so that the (pseudo natural) selection within the oleaginous yeast cells community can occur; and/or so that the most robust oleaginous yeast cells are allowed to propagate and grow further.

By relying on what is described herein, the skilled person is readily able to determine and/or choose how the lignocellulosic preparation is to be added accordingly (e.g. the dosing rate; mode of addition (e.g. continuously, step-wise, bolus-wise, batch-wise)), for example so that the typical growth limitation(s) which would result from a lignocellulose medium being front-loaded (including the abrupt addition of the inhibitor(s)) does/do not occur (or to a substantially lesser extent; e.g. less than 10% or, preferably, 5% thereof). For example, the skilled person is readily able to add the lignocellulosic preparation according to step (ii) so that a limitation of the final oleaginous yeast biomass production level and/or the early-phase yeast biomass production limitation (“early-phase depression”) does/do not occur (or to a substantially lesser extent; e.g. less than 10%, preferably, 5%).

It is preferred that a medium in step (ii) of the inventive methods comprises the same amount(s) (or almost the same amount(s); e.g. at least 60%, 70%, 80% or 90%) and/or the same kind(s)) of inhibitor(s) than the lignocellulose medium to which the oleaginous yeast cells/oleaginous strain is to be adapted in accordance with the invention (e.g. the lignocellulose medium described above) only at the end of step (ii) (optionally including step (ii′)), or at least in the last 50%, 30%, 20% or (preferably) 10% of the duration of step (ii). In the medium in step (ii), the (calculated/added) concentration(s)/amount(s) of the inhibitor(s) thus preferably increase(s) during step (ii) (for example according to a continuous, step-wise, bolus-wise, batch-wise etc. feed of the lignocellulosic preparation which contains the inhibitor(s)). In the medium in step (ii), the concentration of the inhibitor(s) may increase during step (ii) so that the medium in step (ii) reaches (for example at the end of step (ii)) (a) similar (e.g. ±1% to ±20%) or the same (calculated/added) concentration(s)/amount(s) (and/or the same kind) of inhibitor(s) as the lignocellulose medium to which the oleaginous yeast cells/oleaginous strain is to be adapted in accordance with the invention.

The lignocellulosic preparation (containing the carbon source(s) fermentable by an oleaginous yeast and the inhibitor(s)) may be added to the medium according to step (ii) (for example during the (entire) duration of step (ii); optionally including step (ii′)) at an effective glucose dosing rate (or at an overall carbon source(s) dosing rate) between about 0.5 g to about 10 g per kg medium per hour or between about 1.6 g to about 7 g per kg medium per hour or between about 3 g to about 5 g per kg medium per hour (e.g. about 5 g or about 3 g per kg medium per hour); at a dosing rate between about 2 g COD to about 16 g COD per kg medium per hour, between about 3 g COD to about 12 g COD per kg medium per hour or between about 4 g COD to about 10 g COD per kg medium per hour (e.g. about 5 g COD per kg medium per hour); and/or at a dosing rate between about 3 g to about 30 g lignocellulosic preparation per kg medium per hour, between about 5 g to about 20 g lignocellulosic preparation per kg medium per hour or between about 7 g to about 15 g lignocellulosic preparation per kg medium per hour (e.g. about 8.5 g lignocellulosic preparation per kg medium per hour). These adding rates would accordingly determine the adding rates and resulting increase of the inhibitor(s).

The lignocellulosic preparation (containing the at least one carbon source which is fermentable by an oleaginous yeast and the inhibitor(s)) may be added to the medium according to step (ii) at a dosing rate which results in (a) (calculated/added) accumulated concentration(s)/amount(s) (and/or the kind(s)) of the inhibitor(s) in the medium which is (at least at one timepoint during step (ii); e.g. at the end of step (ii), optionally including step (ii′)) similar (e.g. ±1% to ±20%) or the same as in the lignocellulose medium to which the oleaginous yeast cells/oleaginous strain is to be adapted in accordance with the invention.

The lignocellulosic preparation may be added according to step (ii) so that the concentration(s) of the inhibitor(s) linearly increases during the course of step (ii) (preferred), for example from the very beginning to the end of step (ii). This may be achieved by, for example, a linear continuous, linear step-wise, linear bolus-wise etc. addition of the lignocellulosic preparation (for example by a fixed continuous addition rate, fixed frequency of step-wise addition with steps of the same height and fixed frequency of bolus-wise addition with bolus' of the same volumes, respectively). However, the lignocellulosic preparation may, in principle, also be added so that (an) increasing (calculated/added) concentration(s)/amount(s) of the inhibitor(s) occur (for example in cases wherein the actual presence of the inhibitor(s) in the medium is degraded (e.g. by the yeast cells) and/or the yeast cells are particularly sensitive and/or the feed accommodates the (exponential) growth of the yeasts). For example, this can be achieved by an increasing continuous addition, an increasing step-wise addition or an increasing bolus-wise addition of the lignocellulosic preparation. In principle, a decreasing addition rate may also be applied (less preferred).

The lignocellulosic preparation may be added from the very beginning of step (ii) (optionally including step (ii′)) until its end or during only a part of the duration of step (ii), for example during at least 50%, 60%, 70%, 80% or 90% of the duration of step (ii) (optionally including step (ii′)).

The rate of adding the inhibitor(s) usually derive from the adding/feeding rate of the lignocellulosic preparation. In a particular fermentation setting, the adding/feeding rate of the lignocellulosic preparation may accord the carbon source need of the oleaginous yeast cells in the fermenter. This, may result in/determine the adding rate of the inhibitor(s). It is an advantage of the invention that the lignocellulosic preparation can be added according to step (ii) so that the carbon source need of the oleaginous yeast cells is met. Once the adding is like that, the yeast cells adapt to the concomitantly added inhibitor(s) and, thus, to a/the lignocellulose medium.

According to step (ii), the level of (the) concentration(s) of (the) inhibitor(s) as occurring in a/the lignocellulose medium may be reached only partially during the course of step (ii), however, at least to a high degree (e.g. to a degree of at least 60%, 70%, 80%, 90% or (preferably) 100%), provided that the yeast cells still adapt to a/the lignocellulose medium. The resulting (calculated/added) concentration(s) amount(s) of the inhibitor(s) (for example at one timepoint during step (ii); e.g. at the end of step (ii), optionally including step (ii′)) may, for example, be at least 60%, 70%, 80%, 90%, 95% (preferably 100% as compared to the inhibitor in a/the lignocellulose medium.

As mentioned, the inhibitor(s) may be processed and/or degenerated within the medium and/or by the oleaginous yeast cells. If so, the calculated concentration(s)/added amount(s) of the inhibitor(s) in the medium (as added with the lignocellulosic preparation) would exceed the (determinable) concentration actually present in the medium. The addition of the lignocellulosic preparation may be adapted accordingly. In principle, the concentration(s) of the inhibitor(s) mentioned herein pertain to both, the calculated/added concentration(s) (preferred) and the actual (determinable) concentration(s). Especially in cases were extraordinary processing and/or degenerating within the medium and/or by the oleaginous yeast cells occurs or is expected to occur, the concentration(s) of the inhibitor(s) mentioned herein may pertain to the actual (determinable) concentration.

It is preferred that the lignocellulosic preparation (containing the at least one carbon source which is fermentable by an oleaginous yeast and the inhibitor(s)) to be added to the medium according to step (ii) does not constitute (or does not comprise) a “detoxified” lignocellulosic preparation, for example a detoxified lignocellulosic preparation as disclosed in the context of EP2342348B1, WO2010/037780, Liu (loc. cit.), Palmqvist (loc. cit.), Zhang (loc. cit.), US-A1-2016/0002359 and WO2013/122917. However, also “detoxified” lignocellulosic preparations may still contain inhibitor(s) at concentrations which would inhibit/limit the growth of oleaginous yeast cells to a substantial extent (e.g. ≥5% or ≥10%). The use of such “detoxified”, but still inhibiting/limiting lignocellulosic preparations, in the context of step (ii) is also within the scope of the present invention.

In principle, any suitable duration/period (“period” in the following) for step (ii) (optionally including step (ii′)) may be applied in the context of the invention. The skilled person is readily able to choose respective suitable periods, for example for a given fermentation setting. In particular, the skilled person is readily able to choose the period of step (ii) so that the oleaginous yeast cells can adapt in accordance with the invention; for example so that the oleaginous yeast cells are able to undergo metabolic changes necessary to resist the inhibitor(s) present in the lignocellulosic preparation; and/or so that the (pseudo natural) selection within the oleaginous yeast cells community can occur; and/or so that the most robust oleaginous yeast cells are allowed to propagate and grow further; and/or so that an appropriate yield is reached. Periods of feeding phases of typical fed-batch fermentation settings may, for example be applied.

The lignocellulosic preparation may be added during the entire period of step (ii) (optionally including step (ii′)), or only during one (or during several (e.g. at least 2, 3, 4 or 5)) shorter sub-period(s) within the entire period. A sub-period may be about 30%, 40%, 50%, 60% 70%, 80% or 90% of the entire period (the higher values are preferred).

For example, the entire period of step (ii) (optionally including step (ii′), and/or the period during which the lignocellulosic preparation is added to the medium, may be a period of about 10 to about 150 hours, about 10 to about 100 hours, about 20 to about 90 hours, about 30 to about 80 hours. Preferred periods are about 30 to 60 hours, 35 to 55 hours or 40 to 50 (e.g. about 30 hours, 40 hours, 46 hours, 48 hours, 50 hours). It has been shown herein, and in the context of the appended examples, that within such periods, an extraordinary high increase in yeast biomass occurs.

In principle also particularly short and long periods are envisaged (e.g. 20 hours, 10 hours, or even less and 150 hours, 200 hours, or even more, respectively). These particularly short and long periods are, however, less preferred.

Step (ii), optionally including step (ii′), may be conducted until the density of the oleaginous yeast cells in the medium has reached at least 4% (w/w), preferably at least 6% (w/w), more preferably at least 10% (w/w); until an OD₆₀₀ of the yeast cells in the medium of about 60 to 400 or, preferably, of about 100 to 300 has been reached (e.g. an OD₆₀₀ of at least about 200 or at least about 250); and/or until the yeast cells in the medium have reached a dcw of at least about 40 g to 300 g or, preferably, of about 60 g to 200 g per kg medium (e.g. a dcw of about 110 g or about 180 g per kg medium).

In principle, what is said herein above/elsewhere with respect to the (basic) medium and nutrition conditions in step (i) also applies to the medium and nutrition condition in step (ii) (expect the indicated differences which are crucial for the invention (inhibitor(s)). The same applies to step (ii′), expect the lipogenesis induction/increasement (e.g. by nitrogen exhaustion).

In principle, the lignocellulosic preparation to be added according to step (ii) to the medium comprising the yeast cells of step (i) may or may not be the same lignocellulosic preparation which is contained in the lignocellulose medium to which the oleaginous yeast cells/strain are/is to be adapted in accordance with the invention. It is, however, envisaged that the two respective lignocellulosic preparations as providing to the lignocellulose medium the carbon source(s) and inhibitor(s)), i.e. the lignocellulosic preparation to be added in accordance with step (ii) and the lignocellulosic preparation as contained in the lignocellulose medium, share at least the most relevant inhibitor(s) at comparable concentration(s). It is preferred, that the lignocellulosic preparation to be added according to step (ii) is (essentially) the same as the lignocellulosic preparation as comprised in the lignocellulose medium. For example, both, the lignocellulosic preparation to be added according to step (ii) may originate/be derived from the same lignocellulosic material as the lignocellulosic preparation as comprised in the lignocellulose medium (e.g. from the same plant material (e.g. wheat straw)). It is preferred that step (ii) is performed so that and, in particular, that the lignocellulosic preparation is added according to step (ii) so that, during step (ii) (preferably at the end thereof, the lignocellulose medium to which the oleaginous yeast cells are to be adapted in accordance with the invention is formed, in particular in terms of the kind(s) of and (calculated/added) concentration(s)/amount(s) of inhibitor(s) contained.

Any suitable means and methods which induce/increase lipogenesis in oleaginous yeast cells may be applied in the context of (optional) step (ii′) of the methods of the invention (third main step). Respective means/methods are provided herein and by the appended examples. For example, inducing/increasing the lipogenesis in oleaginous yeast cells according to step (ii′) may be achieved by effecting a nitrogen exhaustion/nitrogen deficit. Nitrogen exhaustion/nitrogen deficit may be effected by removal/reduction of the nitrogen feeding regimen. For example, inducing/increasing the lipogenesis in oleaginous yeast cells, nitrogen exhaustion/nitrogen deficit and removal/reduction of the nitrogen feeding regimen, respectively, may be achieved by switching (from a nitrogen-containing pH regulation base (e.g. ammonium hydroxide)) to a nitrogen-free pH regulation base (e.g. sodium hydroxide)). Removal of the relevant (supplements of the) nitrogen feeding would also be an option in this respect.

The oleaginous yeast cells can be harvested according to step (iii) (fourth main step) at an appropriate time point and/or stage of the method/fermentation. For example, the oleaginous yeast cells may be harvested once the density of the yeast cells in the medium has reached from at least about 12% (w/w) to about 15% (w/w); until an OD₆₀₀ of the yeast cells in the medium of about 100 to 300 has been reached (e.g. an OD₆₀₀ of about 200 or about 250); and/or until said yeast cells in the medium have reached a dcw of about 60 to 200 g per kg medium (e.g. a dcw of about 110 or about 180 g per kg medium).

The oleaginous yeast cells (to be) harvested may comprise from between about 40% (w/w) to about 80% (w/w) or (preferably) about 50% (w/w) to about 70% (w/w) fatty acids and/or lipids (including, for example, triacylglycerides).

In accordance with step (iii), the oleaginous yeast cells may be harvested in their entirety, or they may partially be harvested (for example about 5% to 95%, 10% to 90%, 20% to 80%, or 30% to 70% of the cells may be harvested; e.g. (at least) about 95%, 90%, 85%, 80%, 85%, or 70%). A partial harvest may be followed by one (or more) repetition(s) of step (ii) (and, optionally also of step (ii′)). (An) additional step(s) (iii) may also follow.

Means and methods for harvesting oleaginous yeast cells are known in the art (e.g. Yu loc. cit.) and are provided in the appended Examples. Harvesting may comprise centrifugation (e.g. at 5000 rpm or 10000 rpm; e.g. for 5 min), washing the cells or cell pellet (e.g. with (distilled) water, once or at least twice); and/or drying (e.g. at about 100° C. to 105° C.).

After a harvesting step (iii), the fermentation broth (medium, which may still include (same) oleaginous yeast cells) may be recovered/recirculated (including, for example, washing (e.g. with (distilled) water), using diafiltration and/or concentrating (e.g. to a final total solids concentration of from about 35% to about 40%). Recovered fermentation broth may be recirculated into the above-described repetition(s) of step (ii) (and optionally also of step (ii′))

The oleaginous yeast cells at the end of step (ii) (optionally including step (ii′)), and the oleaginous yeast cells which are harvested according to step(s) (iii), constitute the oleaginous yeast strain adapted to a lignocellulose medium in accordance with the invention.

In principle, a method of the invention may be conducted in one or more fermenting device(s).

For example, each of steps (i), (ii) and (optionally) (ii′) may be conducted in a separate fermenting device, or step (i) may be conducted in one fermenting device and step (ii) and (optionally) step (ii′) may be conducted another, separate fermenting device. It is, however, preferred that both, steps (i) and (ii) (and optionally also step (ii′)), are conducted in one single fermenting device. Suitable fermenting devices are well known in the art and are, for example, given in the appended examples. Non-limiting examples of suitable fermenting devices are the Eppendorf-Dasgip parallel bioreactor system (Eppendorf SE, Hamburg, Germany) and (e.g. 3-L) Applikon glass bioreactors.

Any scale of fermentation, and any respective suitable fermenting device, may be used in accordance with the invention (e.g. a propagation fermentation/fermenting device, a seed fermentation/fermenting device and/or a production fermentation/fermenting device). Especially in industrial-scale production processes, (a) production fermentation/fermenting device is/are preferred.

In principle, any kind of fermentation mode may be applied in the context of a method of the invention. Most relevantly, a batch mode, fed-batch mode and/or continuous fermentation mode may be applied. A continuous mode may be a chemostat mode (the feed rate of (a) growth-limiting substance(s) (carbon source(s)) keeps the cell density constant) or a turbidostat mode (the cell density determines the feed rate of (a) substrate(s) (carbon source(s))). It is preferred that step (i) is conducted as a batch/batch phase and/or step (ii) (optionally including step (ii′)), is conducted as a feeding phase (continuous or, preferably, fed-batch). Most preferably, step (i) together with step (ii) (optionally including step (ii′)) are conducted as a fed-batch mode. This means that step (i) is conducted as the batch phase and step (ii) (optionally including step (ii′)) is conducted as the feeding phase. Continuous mode, and especially chemostat mode, is less preferred. Of note, the technical meaning of continuous mode fermentation in this respect does not equal the technical meaning of continuous feeding (like, for example, continuous feeding in the context of a fed-batch mode as may be applied, for example, in the context of the adding according to step (ii)).

In principle, any mode of feeding may be applied in the context of a method of the invention; e.g. bolus-wise feeding (one or more bolus; e.g. 2, 3, 4, 5, 6, 7, 8, 9, or 10 bolus), step-wise feeding (one or more step(s); e.g. 2, 3, 4, 5, 6, 7, 8, 9, or 10 steps), continuous feeding (at a continuous (e.g. linear) feeding rate; this is preferred) and/or at an increasing (or decreasing) feeding rate).

The media to be used in accordance with the invention may already contain the substrate (carbon source(s)) from the very beginning (e.g. in step (i)). This may be with (e.g. preferred for step (ii) or without (e.g. preferred for step (i) additional feeding (e.g. in form of (a) bolus(') or continuous feeding).

In particular, the medium in step (i) may already contain the substrate (carbon source(s)) from the very beginning, with or (preferably) without additional feeding during step (i) (e.g. in form of (a) bolus(')).

The medium in step (ii) may initially contain no substrate (carbon source(s)), or only the residual substrate (carbon source(s)) from step (i) (for example, 0.5% or less of the initial (and/or added) carbon source(s) from the medium in step (i)). In such cases, additional feeding would be required.

In advance of step (i), (an) additional step(s) of a propagation fermentation and/or seed fermentation may be performed (for example for preparing an inoculum for step (i); cf. Examples 1, 3 and 4, infra). (A part of) (the medium comprising) the oleaginous yeast cells cultured by a propagation fermentation and/or seed fermentation may be used as an inoculum for step (i). The size of the inoculum is, in principle, not limiting. Typical inoculum sizes are, however, preferred (e.g. inoculum sizes of about 3%, 5% or 10% v/v IR or w/w (per initial fermenter mass)). Typical inoculums (no lignocellulose-derived carbon source(s) and inhibitor(s)) and their preparations are described in the art (e.g. Niehus loc. cit.; Yu loc. cit.) and in the appended examples (e.g. Examples 1, 3 and 4). It is most preferred that the medium to be used for (the) additional preceding step(s) of a propagation fermentation and/or seed fermentation does not contain a lignocellulose preparation (and carbon source(s) derived therefrom).

Step (i) itself does not constitute a typical inoculum preparation, (a part of) which is then used as an inoculum for (a) subsequent step(s), e.g. for step (ii) and/or as an inoculum for a lignocellulose medium (containing growth-limiting amounts of inhibitor(s)). It is not envisaged according to the invention that (a part of) the cells or cell culture resulting from step (i) is transferred as an inoculum to a lignocellulose medium (for example including the (final) growth-limiting inhibitor(s) amount(s)). This would constitute an abrupt switch form a lignocellulose-free medium to a lignocellulose medium which is not within the scope of the invention. In accordance with this, the volume of the medium in step (i) of the invention is typically larger than the volume of the lignocellulosic preparation (or lignocellulose medium comprising it) to be added during step (ii). This is in contrast to volumes of typical inoculums of the art which are typically smaller that the volumes of the media to be inoculated (even in cases where large inoculums are used; cf. Yu loc. cit.).

The present invention further relates to an oleaginous yeast strain (e.g. R. toruloides strain) adapted to a lignocellulose medium and produced, producible, obtained and/or obtainable by any of the methods of the invention. What is said with respect to these methods, including the lignocellulose medium, herein elsewhere applies here, mutatis mutandis.

The present invention further relates to an oleaginous yeast strain (e.g. R. toruloides strain) adapted to a lignocellulose medium, said strain being characterized by an activated metabolic stress response RPOS (as shown by, for example, transcriptomic sequencing data).

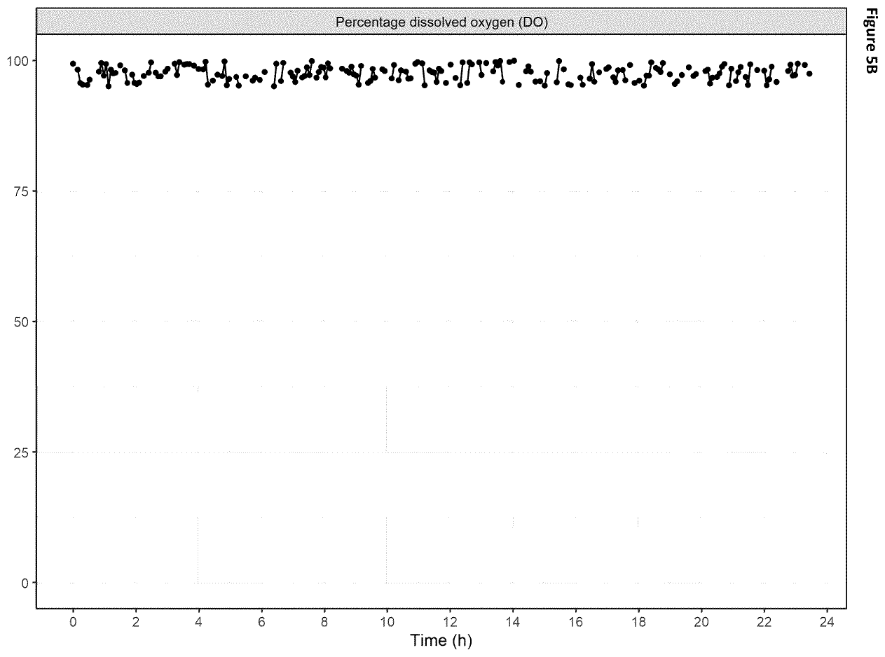

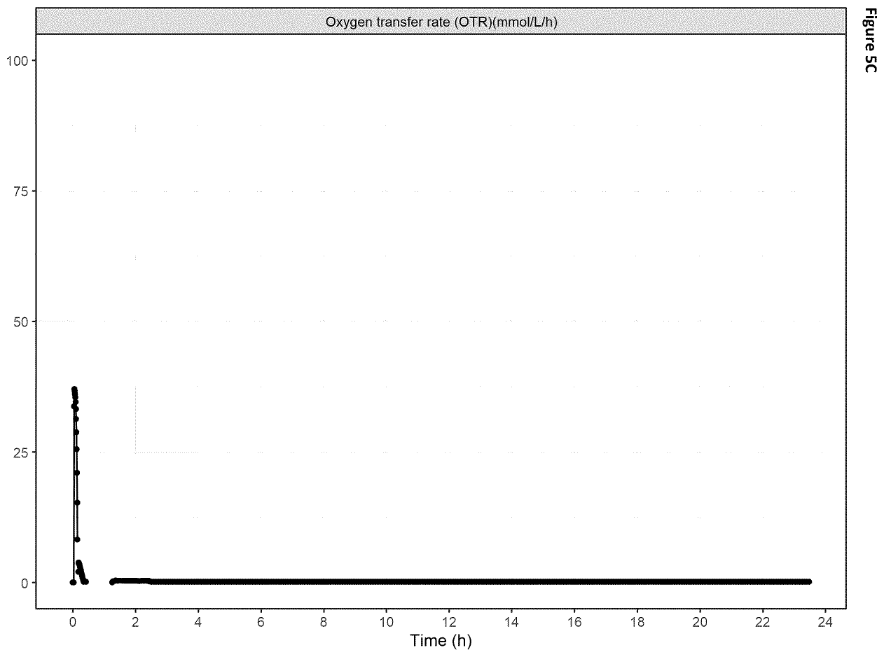

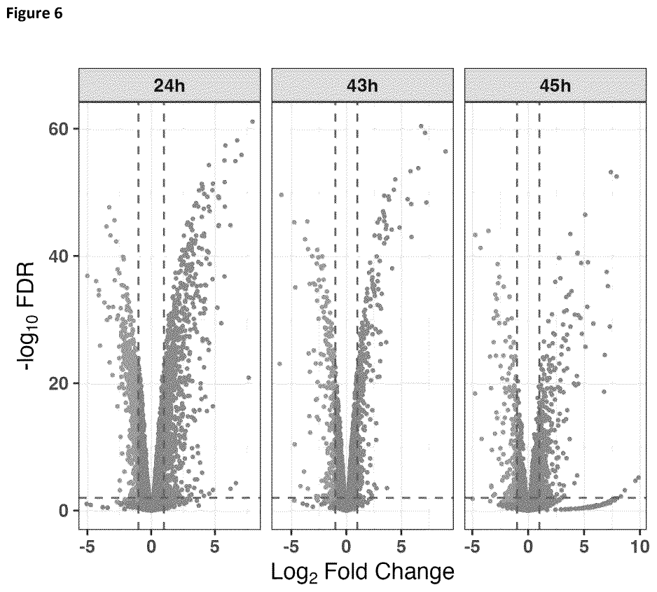

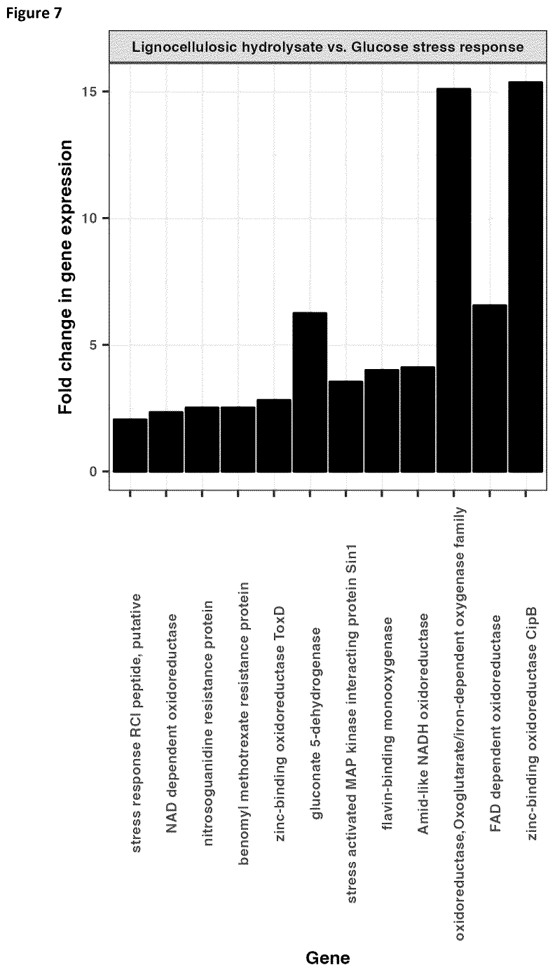

In general, an adapted oleaginous yeast strain of the invention (oleaginous yeast strain adapted to a lignocellulose medium) may be characterized by a particular RNA expression profile (for example as described in appended Example 7, FIG. 6 or FIG. 7).

The present invention thus further relates to an oleaginous yeast strain (e.g. R. toruloides strain) adapted to a lignocellulose medium, said strain being characterized by a particular RNA expression profile (as shown by, for example, transcriptomic sequencing data).

For example, one, two, three, more or all of the genes as depicted in FIG. 7 and/or of the following genes may be upregulated (e.g. on mRNA level) in the adapted oleaginous yeast strain of the invention (e.g. adapted R. toruloides strain):
(i) XM_016418757.1 (encoding zinc-binding oxidoreductase CipB);
(ii) XM_016418495.1 (encoding zinc-binding oxidoreductase ToxD);
(iii) XM_016417115.1 (encoding a gluconate 5-dehydrogenase);
(iv) XM_016421206.1 (encoding a gluconate 5-dehydrogenase); and
(v) XM_016417695.1 (encoding stress activated SIN1 from mitogen activated kinase (MAPK) superfamily).

In addition to the above-mentioned gene(s) (or in the alternative thereto), one, two, three or all of the following genes may (also) be upregulated (e.g. on mRNA level) in the adapted oleaginous yeast strain of the invention (e.g. adapted R. toruloides strain):
(vi) XM_016418081.1 (encoding benomyl methotrexate resistance protein);
(vii) XM_016413927.1 (encoding an oxidoreductase from oxoglutarate/iron-dependent oxygenase family);
(viii) XM_016416917.1 (encoding a NAD dependent oxidoreductase); and
(ix) XM_016420384.1 (encoding a NADH:flavin oxidoreductase/NADH oxidase).

In one embodiment, the present invention relates to an adapted oleaginous yeast strain (e.g. adapted R. toruloides strain) in which at least one, preferably both, of XM_016418757.1 and XM_016418495.1 are upregulated (e.g. on mRNA level). XM_016418495.1 may be upregulated (e.g. on mRNA level) at least 2-fold, preferably at least 5-fold. XM_016418757.1 may be upregulated (e.g. on mRNA level) at least 5-fold, preferably at least 10-fold, more preferably about 15-fold. One, two, three, more or all of the other genes may additionally be upregulated (e.g. on mRNA level).

In one embodiment, the present invention relates to an adapted oleaginous yeast strain (e.g. adapted R. toruloides strain) in which at least one, preferably both, of XM_016418757.1 and XM_016413927.1 are upregulated (e.g. on mRNA level). XM_016413927.1 may be upregulated (e.g. on mRNA level) at least 5-fold, preferably at least 10-fold, more preferably about 15-fold. XM_016418757.1 may be upregulated (e.g. on mRNA level) at least 5-fold, preferably at least 10-fold, more preferably about 15-fold. One, two, three, more or all of the other genes may additionally be upregulated (e.g. on mRNA level).

“Upregulated” in the context of the adapted oleaginous yeast strain of the invention means up-regulated (e.g. on mRNA level; mRNA level is increased) as compared to the/a “control” oleaginous yeast strain (non-adapted), e.g. the oleaginous yeast strain which was grown under pure glucose fermentation conditions (see, for example “control” in Example 1). The “control” oleaginous yeast strain may also be grown under other fermentation conditions, provided that no lignocellulose-derived fermentable carbon source is used (i.e. grown without contact to the respective inhibitors as comprised in a lignocellulosic preparation; i.e. non-adapted oleaginous yeast strain). More particular, “upregulated” in the context of the adapted oleaginous yeast strain of the invention (e.g. on mRNA level) means that the magnitude of fold change is ≥2 (with an adjusted p value of <0.01) as compared to the “control” oleaginous yeast strain. Preferably, the magnitude of fold change is ≥2.5, ≥3, ≥3.5, ≥4, ≥5 or ≥6 (each with an adjusted p value of <0.01).

In the context of the methods and assays for testing the expression of genes in accordance with the invention (e.g. on mRNA level), for example for testing whether a gene is upregulated or downregulated (e.g. mRNA level is increased or decreased, respectively), the difference in transcriptomic response(s) (e.g. based on mRNA level(s) of one or more mRNA(s) transcribed from one or more genes) of the yeast cells/strain to the presence of toxins (“inhibitors”) in lignocellulosic preparation/hydrolysate may be elucidated. Respective cell samples may be (aseptically) collected at the end of step (i) (e.g. end of the batch phase; e.g. EFT^{~} 22 h), and/or, for example, two, twenty one and/or twenty three hours (EFT^{~} 24 h, 43 h and 45 h) after the start of step (ii) (e.g. after the start of fed-batch phase). Cell growth may by arrested by quenching the cell broth (e.g. in a dry ice/ethanol mixture; cf. Example 7). Cell samples may (then) be subjected to transcriptome sequencing and/or mRNA quantification (e.g. by northern blot or (quantitative) reverse transcription polymerase chain reaction (RT-PCR); internal RNA competitive standards (competitors) may be used in this respect). Data, e.g. (m)RNA sequencing data, may be analyzed by aligning, e.g. the raw ((m)RNA) reads, to a publicly available oleaginous yeast transcripts database (e.g. a Rhodotorula toruloides transcripts database; e.g. https://ftp.ncbi.nlm.nih.gov/genomes/all/GCF/000/320/785/GCF_000320785.1_RHOziaDV1.0/GCF_000320785.1_RHOziaDV1.0_rna.fna.gz). NCBI BLAST may be used in this respect (https://blast.ncbi.nlm.nih.gov/Blast.cgi?PROGRAM=blastn&PAGE_TYPE=BlastSearch&LINK_LOC=blasthome). A read count table may be generated by using standard methods. The read count table may be analyzed with statistical software (e.g. R; for example by using the Bioconductor package “edgeR” with glmFIT method). Fold changes in gene expression may be calculated, e.g. with the corresponding p-values. Further, Bonferroni correction may be applied to the p-values to obtain the false discovery rate (FDR). Genes may be considered up- or down-regulated if there was a certain magnitude of fold change (e.g. in mRNA expression); e.g. upregulated if the magnitude of fold change was, for example, ≥2 (e.g. with an adjusted p value of <0.01). Differentially expressed genes/different (up- or downregulated) gene expression (e.g. on mRNA level) for cells growing on lignocellulosic preparation/hydrolysate as the sole/main carbon and energy source, as compared to cells growing on a non-lignocellulose-derived carbon/energy source (e.g. pure glucose), may be determined. Differentially expressed genes/different (up- or downregulated) gene expression (e.g. on mRNA level) may be displayed as a volcano plot. Differentially expressed genes/different (up- or downregulated) gene expression (e.g. on mRNA level) may be tested in a given oleaginous yeast strain; after, for example, 2, 21 and/or 23 hours of exposure to (the toxins of/in) lignocellulosic preparation/hydrolysate (e.g. according to step (ii) described herein). The results may be compared with the (likewise tested) control.

Methods and assays for testing the (upregulation or downregulation of the) expression of genes (e.g. on mRNA level), for RNA expression profiling, and for transcriptomic sequencing, and the like, are known in the art and are also given in the appended Examples (e.g. Example 7). Assaying/testing on mRNA level includes, for example, quantification of the respective mRNA.

In principle, the oleaginous yeast cells or the oleaginous yeast strain used or produced or producible in accordance with the invention may be genetically engineered or non-genetically engineered (the latter is preferred). Further, the oleaginous yeast cells or the oleaginous yeast strain may be monoclonal (i.e. derived from a single oleaginous yeast cell or single oleaginous yeast clone) or polyclonal (i.e. derived from several, genetically different, oleaginous yeast cells or several, genetically different, oleaginous yeast clones).

The present invention further relates to a method of producing oleaginous yeast cells, said method comprising
(I) the steps of
   (i) growing oleaginous yeast cells in a medium which is essentially free of compounds which inhibit the growth of said oleaginous yeast cells and which are derivable from lignocellulosic material (“inhibitors” herein elsewhere), wherein said medium comprises at least one carbon source which is fermentable by said yeast cells (corresponding to the first main step described above);
   (ii) adding to said medium comprising said yeast cells a lignocellulosic preparation (including at least one of said inhibitors), and further growing said oleaginous yeast cells in the medium (corresponding to the second main step described above);
   (ii′) optionally inducing/increasing lipogenesis in said oleaginous yeast cells (corresponding to the third (optional) main step described above); and
   (iii) harvesting said oleaginous yeast cells (corresponding to the fourth main step described above); or
(II) the steps of
   (i) growing an adapted oleaginous yeast strain as produced or producible according to any of the methods of the invention or the adapted oleaginous yeast strain of the invention in a lignocellulose medium (e.g. as defined herein elsewhere) (cf. main step (ii));
   (ii) optionally inducing/increasing lipogenesis in said oleaginous yeast strain (cf. main step (ii′)); and
   (iii) harvesting the cells of said grown oleaginous yeast strain (cf. main step (iii)).

The present invention further relates to a method of producing oils, fatty acids and/or lipids, said method comprising
(I) the steps of
   (i) growing oleaginous yeast cells in a medium which is essentially free of compounds which inhibit the growth of said oleaginous yeast cells and which are derivable from lignocellulosic material (“inhibitors” herein elsewhere), wherein said medium comprises at least one carbon source which is fermentable by said yeast cells (corresponding to the first main step as described above);
   (ii) adding to said medium comprising said yeast cells a lignocellulosic preparation (including at least one of said inhibitors), and further growing said oleaginous yeast cells in the medium (corresponding to the second main step as described above);
   (ii′) optionally inducing/increasing lipogenesis in said oleaginous yeast cells (corresponding to the third main step as described above);
   (iii) harvesting said oleaginous yeast cells (corresponding to the fourth main step as described above); and
   (iv) extracting from said oleaginous yeast cells oils, fatty acids and/or lipids; or
(II) the steps of
   (i) growing an adapted oleaginous yeast strain as produced or producible according to any of the methods of the invention or the adapted oleaginous yeast strain of the invention in a lignocellulose medium (e.g. as defined herein elsewhere) (cf. main step (ii));
   (ii′) optionally inducing/increasing lipogenesis in said oleaginous yeast strain (cf. main step (ii′));
   (ii) harvesting the cells of said grown oleaginous yeast strain (cf. main step (iii)); and
   (iv) extracting from said cells of said grown oleaginous yeast strain oils, fatty acids and/or lipids.

Extracting oils, fatty acids and/or lipids may be performed as known in the art (e.g. Niehus loc. cit.; Yu loc. cit.), or as described in the appended examples.

If not indicated differently herein elsewhere, narrower ranges of values provided herein are preferred over broad ranges. The same applies to particular values (or to narrow ranges) which are covered by the narrower ranges. In principle, what has been said herein elsewhere with respect to the methods of producing an oleaginous yeast strain in accordance with the invention, and with respect to the respective steps, applies mutatis mutandis to the methods of producing oleaginous yeast cells and oils, fatty acids and/or lipids, respectively, as well as to the corresponding steps. In the respective steps (II (i), however, higher amounts of lignocellulosic preparations (including higher amounts of carbon source(s) and inhibitor(s)) may be present or added; as compared to the second main step (step (ii)) (due to the already adapted state of the used oleaginous yeast strain). Likewise, the duration/period of the respective steps (II) (i) may be shorter; as compared to the second main step (step (ii)). For example, the present/added amounts of lignocellulosic preparation (including carbon source(s) and inhibitor(s)) may be higher by a factor of (at least) 1%, 2%, 3%, 5%, 10%, 20% or 30% and/or the duration/period may be shorter y a factor of (at least) 1%, 2%, 3%, 5%, 10%, 20% or 30%.

If not explicitly indicated differently herein elsewhere, terms like “about”, “similar”, “essentially” mean at most +/−5%, +/−3%, +/−2% or +/−1% of the respective reference point (e.g. reference value). It is preferred, however, that the deviation from the reference point is as low as possible.

The present invention further relates to a method of producing (vegan) nutraceuticals/nutritional supplements or petroleum replacers, said method comprising the steps as defined in any one of the methods of the invention.

The present invention is further described by reference to the following non-limiting figures and examples.

The Figures show:

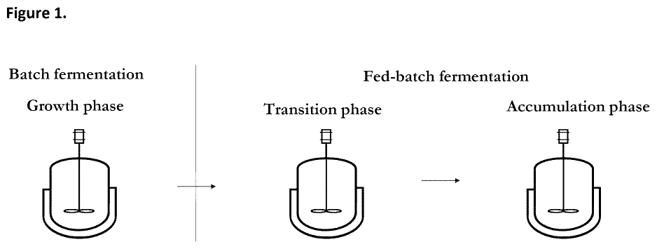

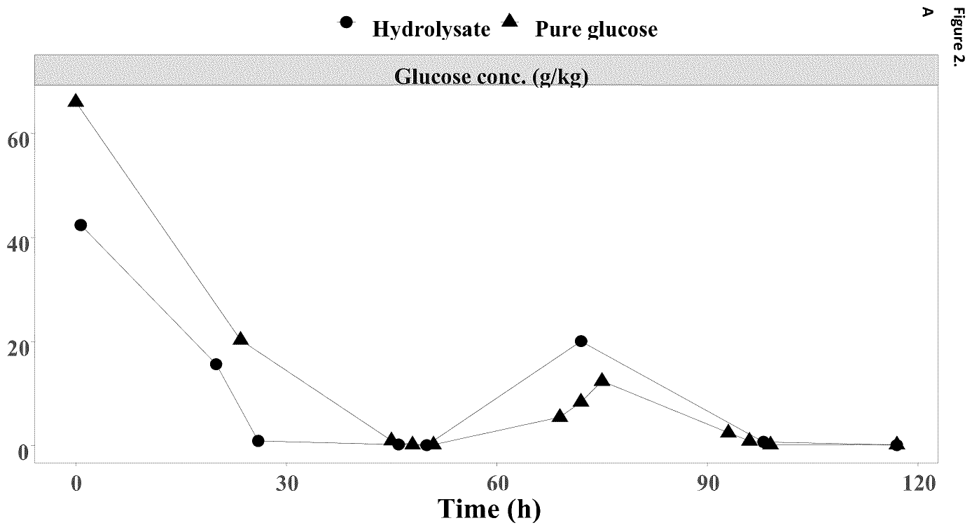

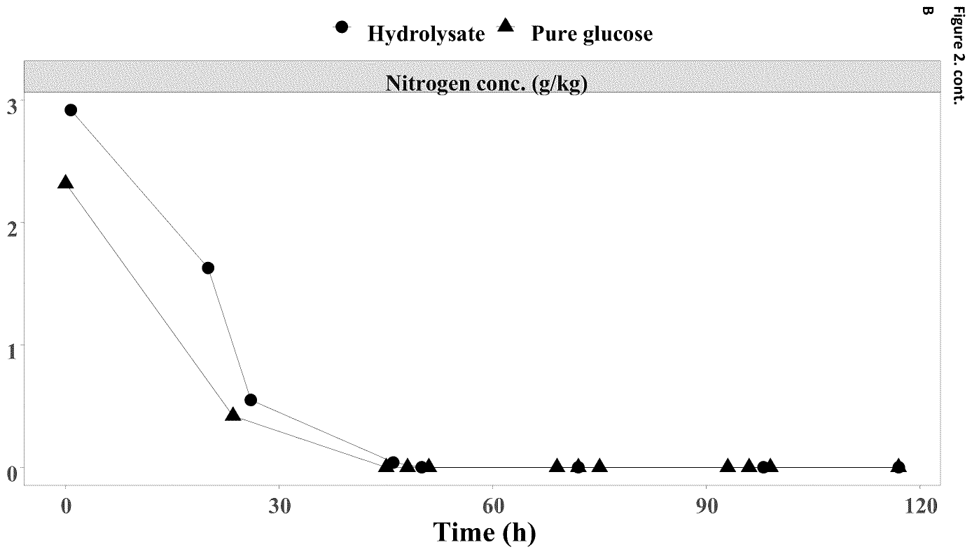

FIG. 1. General, illustrative scheme of a three-stage fermentation process of the invention (including the optional lipid accumulation stage; the scheme is exemplarily shown in the context of a fed-batch operation mode).
Growth phase (first main step (step (i)): Optimal/appropriate growth conditions; pure glucose carbon source (and/or alternative non-lignocellulose-derived carbon source); no inhibitor(s) exposure; establishment of robust yeast community; low lipid accumulation.
Transition phase (second main step (step (ii)): Hydrolysate-derived carbon source (e.g. glucose) is fed slowly; ample/optimal nutrients are available to support cell growth; inhibitor(s) concentration(s) increases gradually in the reactor allowing cells to respond metabolically and continue growing; slow exposure to inhibitor(s) causes a (pseudo-natural) selection within the localized yeast cells community.
Accumulation phase (optional) (third main step (step (ii′)): Hydrolysate-derived carbon source (e.g. glucose) is fed slowly under nitrogen-limited growth conditions; high biomass concentration is built up in the reactor, which is also resistant to inhibitors; cells accumulate excess carbon as lipids/triglycerides.

FIG. 2. R. toruloides fermentation (1^{st} experiment) starting with glucose (non-lignocellulose-derived) as the carbon source (1^{st} main step), followed by fermentation based on lignocellulosic preparation as the carbon source (2^{nd} main step), —comparison with fermentation based on glucose as the sole carbon source

A: Comparison of glucose consumption profile between pure glucose and lignocellulosic glucose as carbon source. B: Comparison of nitrogen consumption profile between pure glucose and lignocellulosic glucose as carbon source. C: Comparison of dry cell weights between pure glucose and lignocellulosic glucose as carbon source. D: Comparison of optical density between pure glucose and lignocellulosic glucose as carbon source. E: Comparison of wet cell weights between pure glucose and lignocellulosic glucose as carbon source. F: Comparison of biomass yield (dry biomass produced per unit glucose supplied) between pure glucose and lignocellulosic glucose as carbon source. G: Comparison of % lipid accumulation in the cells between pure glucose and lignocellulose-derived glucose as carbon source. H: Comparison of lipid titers between pure glucose and lignocellulose-derived glucose as carbon source.

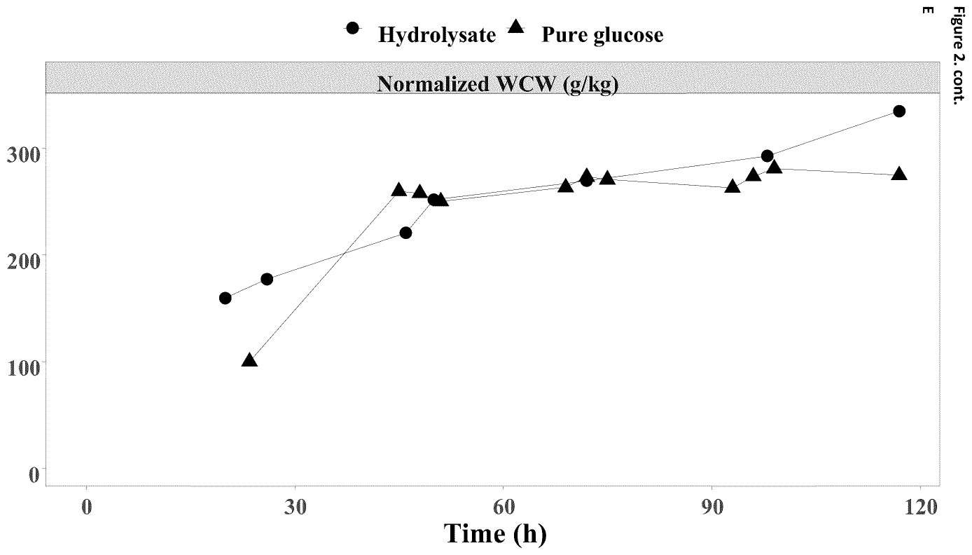

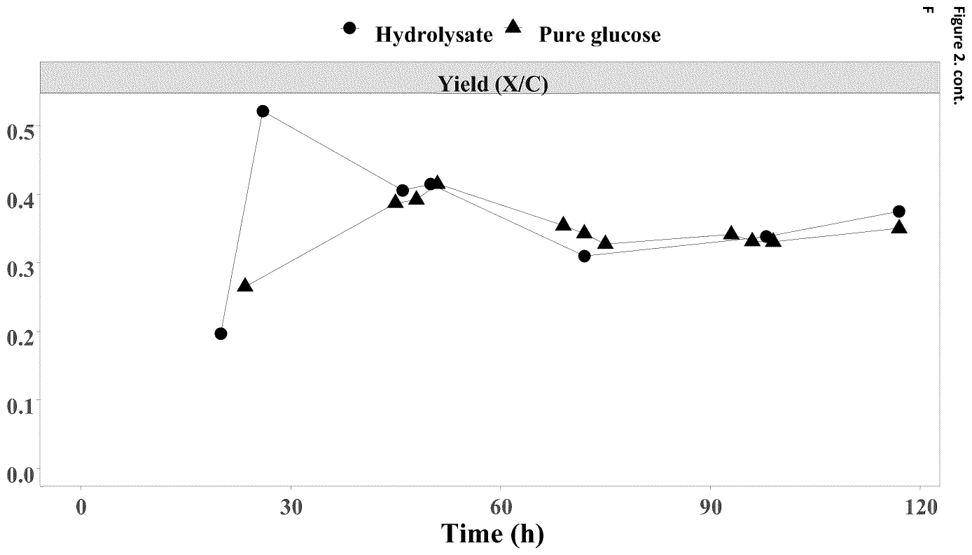

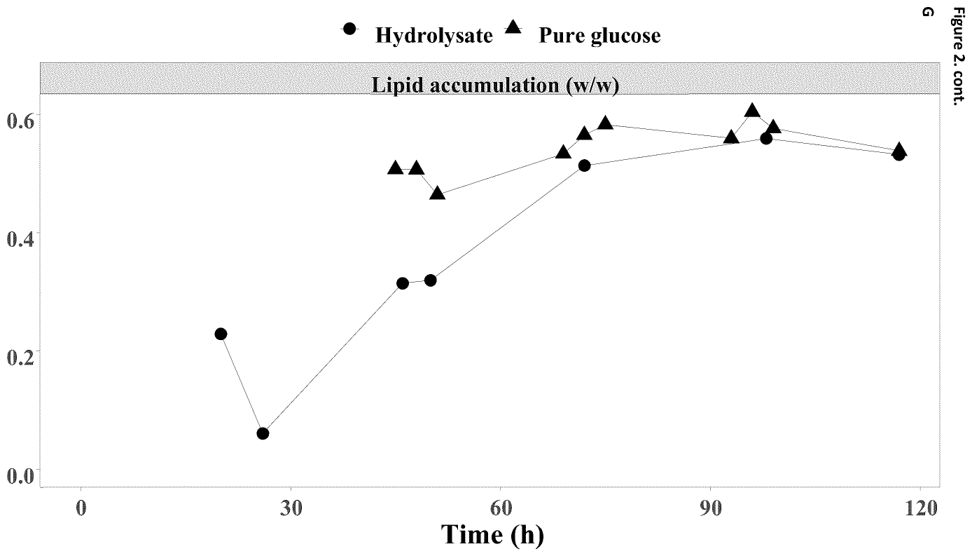

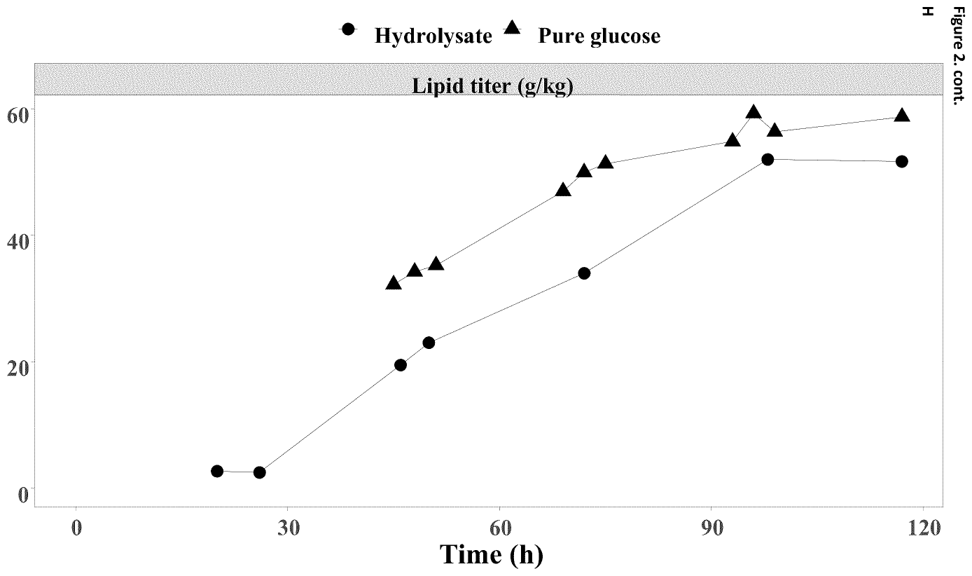

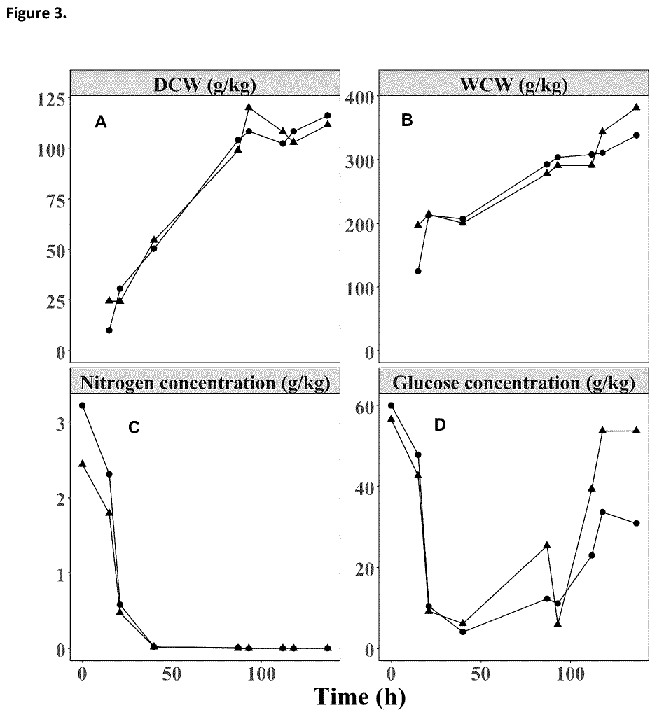

FIG. 3. R. toruloides fermentation (2^{nd} experiment) starting with glucose (non-lignocellulose-derived) as the carbon source (1^{st} main step), followed by fermentation based on lignocellulosic preparation as the carbon source (2^{nd} main step), —comparison with fermentation based on glucose as the sole carbon source

A: Comparison of dry cell weights between pure glucose and lignocellulosic glucose as carbon source. B: Comparison of wet cell weights between pure glucose and lignocellulosic glucose as carbon source. C: Comparison of nitrogen consumption profile between pure glucose and lignocellulosic glucose as carbon source. D: Comparison of glucose consumption profile between pure glucose and lignocellulosic glucose as carbon source.

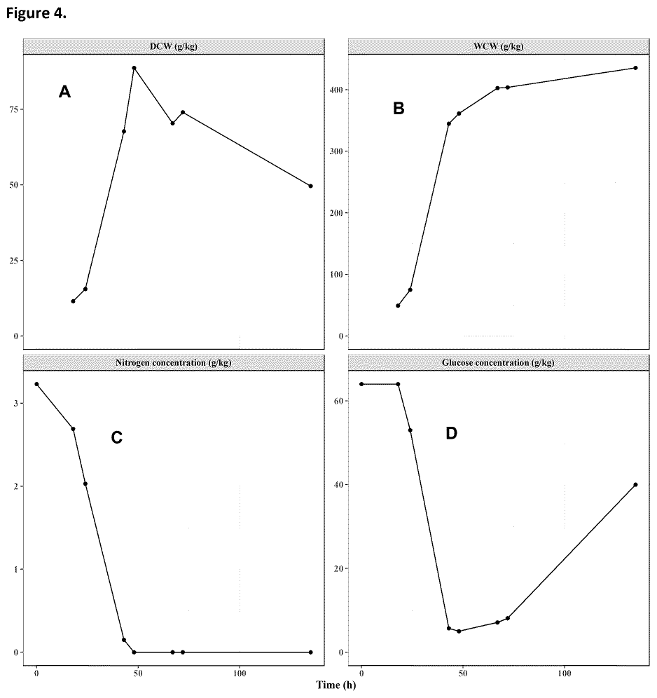

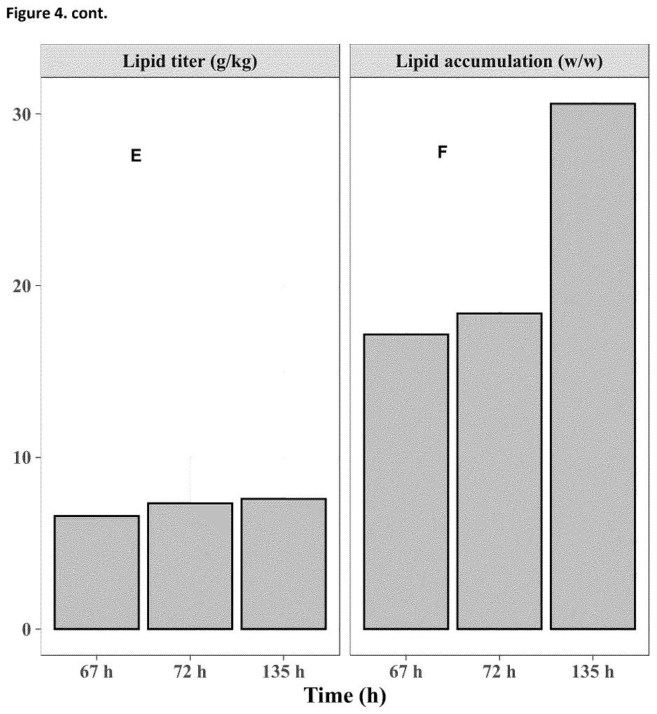

FIG. 4. Y. lipolytica fermentation starting with glucose (non-lignocellulose-derived) as the carbon source (1^{st} step) followed by fermentation based on lignocellulosic preparation as the carbon source (2^{nd} step)—comparison with fermentation based on glucose as the sole carbon source

Except the use of Y. lipolytica as the oleaginous yeast species, the experiment was performed as in Example 3 and FIG. 2, respectively.

A: Biomass density curve as dry biomass weight (dcw). B: Biomass density curve as wet cell weight (wcw). C: Nitrogen concentration. D: Glucose concentration. E: Lipid titer. F: Lipid accumulation.

FIG. 5. Growth inhibition in R. toruloides fermentation (non-adapted strain) when using lignocellulosic preparation as the sole carbon source (negative control)

Fermentation was performed as described in Examples 1 and 3, below, with the exception that also the batch medium contained 60 g per liter of the wheat straw hydrolysate (instead of the pure glucose). A: CER: Carbon evolution rate. B: DO: Dissolved oxygen. C: OTR: Oxygen transfer rate.

FIG. 6. Volcano plots of differential gene expression in adapted R. toruloides strain

The figure shows a snapshot of differentially expressed genes for R. toruloides cells grown on lignocellulosic hydrolysate as the sole carbon and energy source as compared to glucose as the sole carbon and energy source. x-axis represents the magnitude of up- or down-regulation, while the y-axis represents the statistical significance of the difference.

FIG. 7. relative fold changes in the expression of key genes in adapted R. toruloides strain

The figure shows relative fold-changes in the expression of key genes reported to play a role in stress responses of yeast metabolism. Key genes of MAPK stress response superfamily and zinc finger family oxidoreductases were highly upregulated upon exposure to lignocellulosic hydrolysate.

In the foregoing detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention will now be described by reference to the following examples which are merely illustrative and are not to be construed as a limitation of the scope of the present invention.

### EXAMPLE 1

### Materials and Methods (Especially Pertaining to Example 3)

### Microorganism

The Rhodotorula toruloides yeast strain (DSMZ 4444) and the Yarrowia lipolytica yeast strain (DSMZ 70562) used in this study were purchased from Leibniz Institute DSMZ (German collection of microorganisms and cell cultures). The strain was maintained at −80° C. on yeast peptone dextrose (YPD) medium (20 g/L glucose, 20 g/L peptone, and 10 g/L yeast extract) with 15% glycerol (v/v). The culture was further propagated through monthly subcultures on YPD media (described previously and herein elsewhere).

### Lignocellulosic Preparation

As the lignocellulosic preparation, 100% wheat straw hydrolysate was used. Wheat straw hydrolysate was prepared as follows: Wheat straw was pretreated with hydrochloric acid for initial hydrolysis of cellulose and hemicellulose, this was followed by an enzymatic treatment to convert the cellulose into sugars. In the used hydrolysate, the presence of growth inhibitors (furfurals) has been confirmed (at growth-limiting concentrations).

### Media and Batch Culture Conditions

Seed cultures for the fermentation were grown in a baffled 200 mL Erlenmeyer shake flask purchased from VWR Scientific, USA. Shake flask containing 30 mL modified YPD media (30 g/L glucose, 20 g/L peptone, and 10 g/L yeast extract) was inoculated with 1.5 mL frozen glycerol stock. Shake flasks were grown overnight at 30° C. in an Eppendorf Innova S44i shaker (Eppendorf SE, Hamburg, Germany) with a shaker amplitude of 25 mm and a shaker speed of 250 rpm for a total culture time of 23 hours until the glucose was nearly exhausted (<5 g/L) and an OD₆₀₀ of 12 was reached. The exponentially growing cells were then used to inoculate the production reactors. All reactors were operated under fed-batch operation mode and were carried out in Eppendorf Dasgip parallel bioreactor system (Eppendorf SE, Hamburg, Germany) culture vessels with a maximum working volume of 1800 mL. 30 mL seed culture from the shake flask was then used to inoculate (5% v/v IR) the bioreactor containing 600 mL batch media. The batch medium contained per liter: 60 g glucose, 2 g, yeast extract, 0.93 g calcium sulfate dihydrate, 18.2 potassium sulfate, 5 g magnesium sulfate heptahydrate, 6 g citric acid, 20 g potassium phosphate monobasic, 10 g ammonium sulfate, 0.8 g zinc sulfate heptahydrate, 0.2 g sigma 204 antifoam, 0.1 g copper sulfate heptahydrate, 0.05 g manganese sulfate monohydrate, and 1 g ferrous sulfate heptahydrate. All chemicals were purchased from Fisher Scientific GmbH (Schwerte, Germany). The bioreactor containing 600 mL medium was steam sterilized in an autoclave at 121° C. for 20 minutes. The pH of the reactor was controlled at 6+/−0.05 through automated addition of 30% acetic acid or with 30% ammonium hydroxide during the growth and transition phase, and with 10M sodium hydroxide (NaOH) during the lipid accumulation phase. The dissolved oxygen (pO₂) was maintained at a value of 30% of DO saturation at STP through an automated cascade control using air flow, Rushton impellers and pure O₂ dosing.

### Fed-Batch Operation

The end of batch phase was determined by a drop in off-gas CO₂ signal below 1.5% (at this point, the OD₆₀₀ was (about) 60, the WCW was (about) 120 g/kg, the DCW was (about) 30 g/kg, the batch run time was (about) 24 hours (EFT) and the residual glucose concentration was almost zero). At this point (i.e. after a ^{~}24 hours batch phase), a constant feed of 100% wheat straw hydrolysate (obtained from a third party; undisclosed; see herein elsewhere for the production protocol) containing 30% w/w glucose and 7% w/w xylose was started at a rate of 5 g glucose kg⁻¹h⁻¹, which was then maintained for the duration of the fermentation run (EFT 120 hours). As a control, a corresponding glucose feed (non-lignocellulose-derived) was performed in a separate comparable fermentation setting. The lipid accumulation phase was started once the reactor reached an OD₆₀₀ of ^{~}200 and a dry cell weight of ^{~}110 g/kg. The accumulation phase was started upon nitrogen exhaustion in the system (EFT ^{~}72 h) and continued until ^{~}EFT 120 hours. During this phase, the oleaginous biomass continues carbon uptake and stores it intracellularly in the form of triglycerides. Fermentation ended once carbon is no longer consumed by the oleaginous biomass and the intracellular lipid content has reached 50-75% w/w of dry weight of oleaginous biomass. Nitrogen limitation was achieved during the accumulation phase through a replacement of the pH control base from 30% ammonium hydroxide to 10M sodium hydroxide.

### EXAMPLE 2

### Analytical Methods (Especially Pertaining to Example 3)

### Cell Growth and Reactor Performance Measurement

Cell growth was estimated by measuring OD at 600 nm and through dry cell weight (or wet cell weight) measurements. To measure dry cell weight, 5 mL sample was withdrawn aseptically, and 1 mL of broth was transferred to a pre-weighed 15 mL centrifuge tube. The exact mass of the cell broth was recorded and then 14 mL of DI water was added to the centrifuge tube to wash the cell broth of salts and other organic material. The tube was then centrifuged at 10,000×g for 5 minutes and the supernatant was discarded. The resulting pellet was weighed to determine the wet cell weight (WCW) and was then resuspended in 1 mL DI water and transferred to a pre weighed aluminum dish that was then dried to completion in a moisture balance to determine the dry cell weight (DCW) percentage. 2 mL fermentation broth was centrifuged at 13000×g for 2 minutes and the supernatant was collected and used for glucose and ammonium measurement. Glucose concentration was measured using GlucCell fermenter glucose monitoring system and ammonia was measured using an Ion selective electrode from Fisher Scientific GmbH (Schwerte, Germany).

### Lipid Titer Measurement and FAME Analysis

Cell lysis of the lipid-containing yeast cells was performed in isopropyl alcohol (IPA). Briefly, fermentation broth was collected in reweighed lysis tubes containing zirconia beads and was centrifuged at 13000×g for 2 minutes and the supernatant was discarded. The resulting cell paste was dried to completion and dry biomass weight was recorded. The dried biomass was resuspended in IPA with a solvent concentration of 3 mL IPA per gram dried solids (3 mL/g). Cell lysis was performed in the Retsch bead beater (Retsch GmbH, Haan, Germany) at maximum frequency for 15 minutes. Hexane was used as the co-solvent for the liquid-liquid extraction of the lipids from the IPA lysate mixture. Hexane was added in a 2:3 (v/v) ratio (IPA:hexane) and the tubes were left on a shaker overnight to complete the extraction. Extraction solution was then centrifuged at 3300×g for 10 minutes and the resulting supernatant was recovered. The resulting monophasic mixture of IPA and hexane was separated with the addition of 30% (v/v) 0.5 M potassium sulfate (K₂SO₄). Resulting mixture was centrifuged at 3300×g for 10 minutes and the top hexane layer (containing lipids) was collected and transferred to a round bottom flasks and dried under vacuum in a rotary evaporator.

Fatty acid methyl ester (FAME) composition of the purified lipids was determined by diluting the purified lipids in methyl acetate to a final concentration of 5 mg/mL. 20 μL of this solution was mixed with equal volume of TMSH and transesterified to produce methyl esters. The identity of methyl esters was determined using GC-MS using helium as the carrier gas in a DB-FATWAX UI-007 column (Agilent, CA, USA) at a flow rate of 1.1 mL/min. Spectra was acquitted in scan mode in a mass range of 46-400 with EI Ion source and compared to standard C10-C24 FAME mix from Restek (Restek, NJ, USA).

### EXAMPLE 3

### Oleaginous Yeast (R. toruloides) Fermentation (1^{st} Experiment) Starting with Glucose (Non-Lignocellulose-Derived) as the Carbon Source (1^{st} Main Step) Followed by Fermentation Based on Lignocellulosic Preparation as the Carbon Source (2^{nd} Main Step)—Comparison with Fermentation Based on Glucose (Non-Lignocellulose-Derived) as the Sole Carbon Source

Fermentation, monitoring and analyses were performed according to the general scheme as depicted in FIG. 1 and according to Examples 1 and 2, above. Respective particulars which might not explicitly be described in the context of Examples 1 and 2, were as described in Example 4 (see also, for example, Tables 1, 2, 3, 4, 5, 6), or as described herein elsewhere or in the art.

As shown in FIG. 2, the fermentation performance, biomass and lipid production based on a three-step protocol according to the invention (steps (i), (ii) and (ii′)) equal those based on glucose (non-lignocellulose-derived) as the sole carbon source. In particular, FIG. 2 shows that carbon uptake was not impacted by the presence of the inhibitors in the adapted cells (A), that nitrogen uptake was not impacted by the presence of the inhibitors in the adapted cells (B), and that cell growth was not impacted by the presence of the inhibitors in the adapted cells (C, D, E, F). Further, FIG. 2 shows that adapted cells show similar levels of lipid accumulation and lipid titers between pure glucose (non-lignocellulose-derived) and lignocellulose-derived glucose as the (main) carbon source (G, H).

### EXAMPLE 4

### Oleaginous Yeast (R. toruloides) Fermentation (2^{nd} Experiment) Starting with Glucose (Non-Lignocellulose-Derived) as the Carbon Source (1^{st} Main Step) Followed by Fermentation Based on Lignocellulosic Preparation as the Carbon Source (2^{nd} Main Step)— Comparison with Fermentation Based on Glucose (Non-Lignocellulose-Derived) as the Sole Carbon Source

Fermentation, monitoring and analyses were performed according to the general scheme as depicted in FIG. 1 and as described below. Respective particulars which might not explicitly be described below, were as described in Examples 1, 2 and/or 3 (see also, for example, Tables 1, 2, 3, 4, 5, 6), or as described herein elsewhere or in the art.

### Fermentation Process Overview

A three-stage fermentation process according to the invention was performed (steps (i), (ii) and (ii′)) which utilizes lignocellulosic hydrolysates to deliver lipid productivities comparable to pure glucose fermentation, and without the need for toxin removal or any genetic modifications. The three main stages were growth stage, transition and adaptation stage and lipid accumulation stage.

The three-stage fermentation process was started by propagating a Rhodotorula toruloides strain (DSMZ 4444) in shake flasks, followed by a pure glucose-based growth stage in a fermenter lasting ^{~}23-27 hours. Carbohydrates (primarily glucose) derived from hydrolysis of lignocellulosic feedstock were used as the carbon source during the two fed batch phases, and a lignocellulosic hydrolysate feed to maintain a constant max. OTR value of 90 mmol/L/h was started at the end of the batch phase. The OTR of 90 mmol/L/h was maintained by the subsequently described feed strategy and by adjusting the initial pressure to 800 mbar overpressure, and 1 vvm aeration. The pH was controlled to 6±0.05 by automated addition of 30% ammonium hydroxide during the growth stage and the transition stage, and 10M sodium hydroxide during accumulation phase or 30% acetic acid, and the temperature and the DO were controlled to 32° C. and 30% of saturation at STP, respectively. Samples were taken throughout the fermentation, and the OD₆₀₀, wet cell weight, dry cell weight, residual nitrogen and residual hydrolysate were measured.

### Inoculum Preparation

The inoculum was prepared by cultivation in YPD media in a shake flask. 1.5 mL of frozen glycerol stock was sterilely added to a baffled 500 mL shake flask containing 50 mL of sterile YPD media and the shake flask was cultivated at 32° C. and 350 rpm in an Infors HT Multitron Pro shaker with a shaker amplitude of 25 mm for between 23-25 hours, reaching a target WCW of 60 g/kg and an OD600 of ^{~}30. Table 2 presents the shake flask cultivation conditions.

### Three-Stage Fermentation

1. Growth phase: Once the shake flask propagation was completed, having reached an approximate biomass density of ^{~}60 g/kg WCW in ^{~}23-25 hours, 100 g/kg (10% w/w) of inoculum per initial fermenter mass was added to the production fermenter. The batch phase of the production fermenter lasted ^{~}26-30 hours EFT, reaching an OD₆₀₀ of ^{~}60 and a wet cell weight of ^{~}120 g/kg.
2. Transition phase: A constant lignocellulosic hydrolysate feed at the rate of 5 g-COD/kg/h was triggered automatically at the end of the batch phase by a drop in CO₂ signal, which was then maintained for the duration of the fermentation run. The transition phase lasted for an additional ^{~}48 hours, at the end of which the fermenter reaches an OD₆₀₀ of ^{~}250 and a dry cell weight of ^{~}180 g/kg.
3. Lipid accumulation phase: At the end of the transition phase, the pH regulation base was switched from ammonium hydroxide to sodium hydroxide, which resulted in a nitrogen deficit in the bioreactor. The accumulation phase started upon nitrogen exhaustion in the system and continued until approximately EFT 120 hours. During this phase, the oleaginous biomass continued carbon uptake and stored it intracellularly in the form of triglycerides. Fermentation ended once the carbon was no longer consumed by the oleaginous biomass and the intracellular lipid content has reached 50-75% w/w of dry weight of oleaginous biomass. Tables 3-5 present the composition of the media, stock solutions and batch conditions, and Table 6 presents the production fermentation.

As shown in FIG. 3, the fermentation performance and biomass production based on a three-step protocol according to the invention (steps (i), (ii) and (ii′)) equal those based on glucose (non-lignocellulose-derived) as the sole carbon source. In particular, FIG. 3 shows that cell growth was not impacted by the presence of the inhibitors in the adapted cells (A, B); that nitrogen uptake was not impacted by the presence of the inhibitors in the adapted cells (C); and that carbon uptake was not impacted by the presence of the inhibitors in the adapted cells (D).

### EXAMPLE 5

### Oleaginous Yeast (Y. lipolytica) Fermentation Starting with Glucose (Non-Lignocellulose-Derived) as the Carbon Source (1^{st} Main Step) Followed by Fermentation Based on Lignocellulosic Preparation as the Carbon Source (2^{na} Main Step)—Comparison with Fermentation Based on Glucose (Non-Lignocellulose-Derived) as the Sole Carbon Source

Fermentation was performed as in Example 3, above. Y. lipolytica (strain DSMZ 70562) was used as the oleaginous yeast (instead of R. toruloides).

As shown in FIG. 4, the Y. lipolytica fermentation performance, biomass and lipid production based on a three-step protocol according to the invention ((steps (i), (ii) and (ii′)); cf. FIG. 1) equal those which were achieved for R. toruloides in the context of Examples 3 and 4 (cf. FIGS. 2 and 3). Likewise, FIG. 4 shows that adapted Y. lipolytica cells grown on lignocellulose-derived glucose as the (main) carbon source show high levels of biomass production, lipid accumulation and lipid titers (FIG. 4 A, B, E, F; like cells of R. toruloides grown on pure glucose (non-lignocellulose-derived) and on lignocellulose-derived glucose).

### EXAMPLE 6

### Growth Inhibition in R. toruloides Fermentation (Non-Adapted Strain) when Using Lignocellulosic Preparation as the Sole Carbon Source (Negative Control)

Fermentation was performed as described in Examples 1 and 3, above, with the exception that also the batch medium contained 60 g per liter of the wheat straw hydrolysate (instead of the pure glucose).

As shown in FIG. 5, growth, fermentation performance and biomass production were inhibited completely when fermentation was started with lignocellulosic preparation as the sole carbon source.

### EXAMPLE 7

### RNA Expression Profiling by Transcriptome Sequencing of an R. toruloides Strain Resulting from Fermentation Starting with Glucose (Non-Lignocellulose-Derived) as the Carbon Source (1^{st} Main Step) Followed by Fermentation Based on Lignocellulosic Preparation as the Carbon Source (2^{nd} Main Step) (Adapted R. toruloides Strain)

Rhodotorula toruloides was cultured under the experimental conditions as described above using lignocellulosic hydrolysate as the sole carbon source during the fed-batch phase (see Examples 1 and 3, above) and using pure glucose as the sole carbon source, which served as the negative control (see above Example 1; “control”), to elucidate the difference in transcriptomic responses of the yeast to toxins (“inhibitors”) present in the lignocellulosic hydrolysate.

Cell samples were aseptically collected at the end of the batch phase (EFT=22 h), and, two, twenty one and twenty three hours after the start of the fed-batch phase (EFT=24 h, 43 h and 45 h). Cell growth was arrested by quenching the cell broth in dry ice/ethanol mixture. Cell samples were then sent for transcriptome sequencing.

RNA sequencing data were analyzed by aligning the raw RNA reads to a publicly available Rhodotorula toruloides transcripts database: (https://ftp.ncbi.nlm.nih.gov/genomes/all/GCF/000/320/785/GCF_000320785.1_RHOziaDV1.0/GCF_000320785.1_RHOziaDV1.0_rna.fna.gz) using NCBI BLAST (https://blast.ncbi.nlm.nih.gov/Blast.cgi?PROGRAM=blastn&PAGE_TYPE=BlastSearch&LINK_LOC=blasthome).

A read count table was generated using standard methods. The read count table was then analyzed in statistical software R using the Bioconductor package “edgeR” with glmFIT method to calculate fold changes in gene expression with the corresponding p-values. Further, Bonferroni correction was applied to the p-values to obtain the false discovery rate (FDR). Genes were then considered up- or down-regulated if the magnitude of fold change was >=2 and if the adjusted p value was <0.01.

FIG. 6 shows volcano plots of differential gene expression showing a snapshot of differentially expressed genes for cell growing on lignocellulosic hydrolysate as the sole carbon and energy source compared to glucose.

The comparison of transcriptomes after two hours of exposure to the toxins in lignocellulosic hydrolysate revealed that a total of 2575 genes were differentially expressed, with 1574 genes being up-regulated, while 1001 genes being down-regulated.

Among many highly induced expressions, two zinc finger family oxidoreductase genes, XM_016418757.1 encoding zinc-binding oxidoreductase CipB and XM_016418495.1 encoding zinc-binding oxidoreductase ToxD, were also noticeable in their response to the challenge of the toxins (“inhibitors”).

Among other genes responses of note, both copies of gluconate 5-dehydrogenase encoding genes XM_016417115.1 and XM_016421206.1, gene XM_016417695.1 encoding the stress activated gene SIN1 from mitogen activated kinase (MAPK) superfamily were also upregulated. Genes with strong response to other toxins such as phenolic aldehydes, vanillin and syringaldehyde (“inhibitors”) on the transcriptional level were also screened and four genes, XM_016418081.1, XM_016413927.1, XM_016416917.1 and XM_016420384.1, encoding benomyl methotrexate resistance protein, an oxidoreductase from oxoglutarate/iron-dependent oxygenase family, a NAD dependent oxidoreductase and a NADH:flavin oxidoreductase/NADH oxidase, respectively, were also found to be upregulated.

FIG. 7 shows relative fold changes in the expression of key genes reported to play a role in stress responses of yeast metabolism. Key genes of MAPK stress response superfamily and zinc finger family oxidoreductases were highly upregulated upon exposure to lignocellulosic hydrolysate.

Reference is further made herein to the following table(s):

TABLE 1
Projected final fermenter weight per kg initial fermenter media.

| Component | Weights (g) |
|---|---|
| Total initial weight | 1000 |
| Inoculum (10% IR) | 100 |
| Hydrolysate feed | 432 |
| 30% Ammonium Hydroxide | 40 |
| 10M Sodium Hydroxide | 100 |
| 30% acetic acid | 30 |
| Total final weight | 1702 |

TABLE 2
Shake flask seed propagation conditions.

| Parameter | Value | |
|---|---|---|
| Temperature | 32° | C. |
| Shaker Speed | 350 | rpm |
| Shaker throw (amplitude) | 25 | mm |
| Target final biomass density (OD₆₀₀) | 25 | |
| Target final wet cell weight | 60 | g/Kg |

TABLE 3
Fermenter media composition.

| | Concentration in batch |
|---|---|
| Component | media (g/kg) |
| Pure glucose | 60 |
| Yeast extract | 2 |
| Calcium sulfate dihydrate | 0.93 |
| Potassium sulfate | 18.2 |
| Magnesium sulfate heptahydrate | 5 |
| Citric acid | 6 |
| Potassium phosphate monobasic | 22 |
| Ammonium sulfate | 10 |
| Zinc sulfate heptahydrate | 0.758 |
| Antifoam | 0.2 |
| Copper sulfate pentahydrate | 0.084 |
| Manganese sulfate monohydrate | 0.042 |
| Ferrous sulfate heptahydrate | 0.913 |

Note:
Media at 121 C. for 30 minutes; a small amount of precipitate post sterilization is typical.

TABLE 4
Estimated volume of acid and base required
based on initial fermenter weight.

| Component | Approx. use rate (mL/kg) |
|---|---|
| 10M sodium hydroxide | 100 |
| 30% Ammonium Hydroxide | 40 |
| 30% acetic acid | 30 |

TABLE 5
Glucose and Hydrolysate feed based on initial fermenter volume.

| Component | Concentration (g/kg) |
|---|---|
| Glucose | 600 |
| Hydrolysate | 100% |

Note:
Feed was sterilized at 121 C. for 20 minutes

TABLE 6
Production fermenter feeding profile -
Glucose (Ex. 3)/Hydrolysate (Ex. 4/5).

| | Feed rate (g hydrolysate/kg | Feed rate (g glucose/kg |
|---|---|---|
| Phase | fermenter/hour) | fermenter/hour) |
| Batch phase | 0.0 | 0.0 |
| Fed-batch phase | 8.5 | 5.1 |

TABLE 7
Analysis of saccharified slurries from deacetylated
(P120927DCS) and non-deacetylated (P120927CS) corn
stover (examples of lignocellulosic preparations)
using high-performance liquid chromatography (HPLC),
GC-MS, and LC-DAD-MS (derived from Yang loc. cit.)

| Tentative | | P120927CS | P120927DCS |
|---|---|---|---|
| compound | Detection | conc. | conc. |
| ID | method | (g/L) [symbol] | (g/L) [symbol] |
| C [symbol] | HPLC | 3. [symbol] | [symbol] .7 [symbol] |
| Glu [symbol] | | [symbol] .73 | 99. [symbol] |
| [symbol] | | [symbol] 9. [symbol] | [symbol] 1. [symbol] 3 |
| G [symbol] | | 3.31 | [symbol] .94 |
| A [symbol] | | 5. [symbol] | [symbol] .79 |
| Acetic Acid | | [symbol] | [symbol] .2 |
| F [symbol] | | 0. [symbol] | 0.17 |
| Acetic acid | GC-MS | [symbol] | 0. [symbol] |
| HMF | | [symbol] | 0.16 |
| p-Co [symbol] acid | LC-DAD-MS | 0.1 | 0.12 |
| [symbol] acid | | 0.0 [symbol] | 0.09 |
| V [symbol] | | 0.0 [symbol] | 0.0 [symbol] |
| C [symbol] and [symbol] acid | LC-DAD-MS | 2.07 | [symbol] .2 [symbol] |
| C [symbol] low MW [symbol] + | | 1.22 | 0. [symbol] |
| fatty acids | | | |
| [symbol] | | 1. [symbol] 1 | 1.1 [symbol] |
| Unid [symbol] | | 0. [symbol] | 0. [symbol] |
| Unid [symbol] | | 0.6 [symbol] | 0. [symbol] |
| C [symbol] acids | | 0.4 [symbol] | 0. [symbol] |
| A [symbol] | | 0.07 | 0.06 |
| A [symbol] | | 0.0 [symbol] | 0.0 [symbol] |
| [symbol] acid | | 0.0 [symbol] | 0.02 |
| 4 [symbol] | | 0.01 | [symbol] .01 |
| [symbol] | GC-MS | 0.1 [symbol] | 0.1 |
| [symbol] | | 0.0 [symbol] | 0. [symbol] |
| [symbol] | | 0.019 | 0.00 [symbol] |
| [symbol] | | 0.018 | 0.01 |
| [symbol] | | 0.01 | 0.014 |
| [symbol] | | 0.008 | 0.00 [symbol] |
| [symbol] | | [symbol] .00 [symbol] | 0.00 [symbol] |

[symbol]

[symbol]

indicates data missing or illegible when filed

1. A method of producing an oleaginous yeast strain adapted to a lignocellulose medium, said method comprising the steps of
(i) growing oleaginous yeast cells in a medium which is essentially free of compounds which inhibit the growth of said oleaginous yeast cells and which are derivable from lignocellulosic material, wherein said medium comprises at least one carbon source which is fermentable by said yeast cells;
(ii) adding to said medium comprising said yeast cells a lignocellulosic preparation including one or more compound(s) which inhibit(s) the growth of said oleaginous yeast cells and which are derivable from lignocellulosic material, and further growing said oleaginous yeast cells in the medium; and
(iii) harvesting said oleaginous yeast cells.

2. The method of claim 1, said method further comprising the step of (ii′) inducing/increasing lipogenesis in said oleaginous yeast cells.

3. The method of claim 2, wherein said step (ii′) is performed after step (ii) and before step (iii).

4. The method of claim 1, wherein:
the medium in step (i) comprises:
(a) between from about 30 g to about 120 g of said fermentable carbon source per kg of medium;
(b) about 40 g, 60 g or 80 g of said fermentable carbon source per kg of medium; and/or
(c) as said fermentable carbon source glucose/dextrose, isoglucose, xylose, fructose, sucrose, or DE95; or a mixture of two or more of glucose/dextrose, isoglucose, xylose, fructose, sucrose, or DE95; and/or
step (i) is conducted:
(a) in a time from about 10 hours to about 48 hours;
(b) in a time of about 25, 28 or 30 hours;
(c) until at least about 95% of said carbon source has been consumed by said yeast cells; and/or
(d) until at least about 20% to 40% of the final expected yeast cell biomass has been reached in said medium; until an OD600 of said yeast cells in said medium of about 40 to 80 has been reached; until said yeast cells in said medium have reached a wcw of about 60 to 240 g per kg medium; until said yeast cells in said medium have reached a dcw of about 20 to 50 g per kg medium; until dissolved oxygen (pO2) reached a value of >50% of DO saturation; and/or until a drop in off-gas CO2 signal to <1.5% occurs; and/or
(e) is conducted until at least about 20% to 35% of the final expected yeast cell biomass has been reached in said medium; wherein an OD600 of said yeast cells in said medium of about 60 has been reached; wherein said yeast cells in said medium have reached a wcw of about 120 g per kg medium; and/or wherein said yeast cells in said medium have reached a dcw of about 30 to 40 g per kg medium or about 35 g per kg medium.

5-10. (canceled)

11. The method of claim 10, wherein said final expected yeast cell biomass is determined on the basis of the OD₆₀₀, the wet cell weight (wcw) and/or the dry cell weight (dcw) of said yeast cells in said medium (w/w).

12. (canceled)

13. The method of claim 1, wherein the lignocellulosic preparation:
(a) is added at an effective glucose dosing rate between about 1.6 g to about 7 g per kg medium per hour; at a dosing rate between about 3 g to about 12 g COD per kg medium per hour; and/or at a dosing rate between about 5 g to about 20 g lignocellulosic preparation per kg medium per hour;
(b) is added at an effective glucose dosing rate of about 5 g or about 3 g per kg medium per hour; at a dosing rate of about 5 g COD per kg medium per hour; and/or at a dosing rate of about 8.5 g lignocellulosic preparation per kg medium per hour;
(c) is added over a period of about 20 to about 90 hours or about 30 to 60 hours;
(d) is added over a period of about 30 hours, 40 hours, 46 hours, 48 hours or 50 hours;
(e) does not comprise more than 25% of a non-lignocellulosic carbon source;
(f) does not comprise more than 25% of non-lignocellulose-derived glucose/dextrose, isoglucose, xylose, fructose, sucrose, or DE95; or a mixture of two or more of non-lignocellulose-derived glucose/dextrose, isoglucose, xylose, fructose, sucrose, or DE95;
(g) comprises at least 75% of a lignocellulosic carbon source;
(h) comprises at least 75% of a lignocellulosic carbon source being lignocellulose-derived glucose, xylose and/or arabinose;
(i) comprises about 100% of a lignocellulosic carbon source;
(j) comprises about 30% w/w lignocellulose-derived glucose and about 7% w/w lignocellulose-derived xylose; and/or
(k) comprises one or more of the compounds selected from the group consisting of:
(i) furfural/furfurals;
(ii) 5-hydroxymethyl-furfural (HMF);
(iii) acetic acid;
(iv) formic acid;
(v) levulinic acid; and
(vi) syringaldehyde;
(vii) p-hydroxybenzaldehyde (PHB); and
(viii) vanillin.

14-23. (canceled)

24. The method of claim 1, wherein step (ii′) comprises removal/reduction of the nitrogen feeding regimen.

25. The method of claim 24, wherein the nitrogen feeding regimen comprises:
(a) switching to a nitrogen-free pH regulation base; and/or
(b) switching to a nitrogen-free pH regulation base sodium hydroxide.

26. (canceled)

27. The method of claim 1, wherein step (ii), and optionally step (ii′), is conducted:
(a) until the density of said yeast cells in the medium has reached at least 6% (w/w); until an OD₆₀₀ of said yeast cells in the medium of about 100 to 300 has been reached; and/or until said yeast cells in the medium have reached a dcw of about 60 to 200 g per kg medium; and/or
(b) until the density of said yeast cells in the medium has reached at least 10% (w/w); until an OD600 of said yeast cells in the medium of about 200 or about 250 has been reached; and/or until said yeast cells in the medium have reached a dcw of about 110 or about 180 g per kg medium.

28. (canceled)

29. The method of claim 1, wherein:
(a) said yeast cells are harvested according to step (iii) once the density of said yeast cells in the medium has reached a range from about 12% (w/w) to about 15% (w/w); until an OD₆₀₀ of said yeast cells in the medium of about 100 to 300 has been reached; and/or until said yeast cells in the medium have reached a dcw of about 60 to 200 g per kg medium;
(b) said yeast cells are harvested according to step (iii) until an OD600 of said yeast cells in the medium of about 200 or about 250 has been reached; and/or until said yeast cells in the medium have reached a dcw of about 110 or about 180 g per kg medium;
(c) the harvested yeast cells comprise from between about 50% (w/w) to about 70% (w/w) fatty acids and/or lipids;
(d) said yeast cells in step (iii) are partially harvested followed by one or more repetition(s) of step (ii), and, optionally, also of step (ii′); and/or
(e) the yeast is of a genus selected from the group consisting of Rhodotorula (e.g. R. toruloides, R. babjevae, R. diobovata, R. glutinis, R. mucilaginosa, R. kratochvilovae), Yarrowia (e.g. Y. lipolytica), Cutaneotrichosporon (e.g. C. curvatus, C. guehoae), Naganisha (e.g. albida), Cryptococcus (e.g. C. ramirezgonezianus, C. terricola, C. curvatus), Solicoccozyma (e.g. S. phenolicus), Papiliotrema (e.g. P. baii), Myxozyma (e.g. M. mucilagina, M. melibiosi), Vishniacozyma (e.g. V. aff. heimaeyensis), Vanrija (e.g. V. musci), Leucosporidium (e.g. L. scottii), Scheffersomyces (e.g. S. stipitis), Lipomyces (e.g. L. starkeyi), Candida (e.g. C. insectorum), Filobasidium (e.g. F. magnum, F. wieringae), Rhodosporidium (e.g. R. fluvialis), Metschnikowia (e.g. M. pulcherrima), Wickerhamomyces (e.g. W. siamensis).

30-33. (canceled)

34. The method of claim 1, wherein, after the harvesting step (iii), the fermentation broth is recovered/recirculated optionally including washing with water using diafiltration and concentrating to a final total solids concentration of from about 35% to about 40%.

35. (canceled)

36. The method of claim 1, wherein
(a) steps (i) and (ii), and, optionally, also step (ii′), are conducted in one single fermenting device (e.g. a production fermenting device);
(b) step (i) is conducted as a batch/batch phase; and/or
(c) step (ii), and, optionally, also step (ii′), or steps (i) and (ii), and, optionally, also step (ii′), is/are conducted as a fed-batch/fed-batch phase.

37. The method of claim 1, wherein steps (i) and (ii), and, optionally, also step (ii′), are conducted in a production fermenting device.

38. An oleaginous yeast strain adapted to a lignocellulose medium, wherein said oleaginous yeast strain is:
(a) produced by the method of claim 1;
(b) characterized by an activated metabolic stress response RPOS and MAPK superfamily;
(c) characterized by an activated metabolic stress response RPOS and MAPK superfamily as shown by transcriptomic sequencing data;
(d) characterized by a specific RNA expression profile; and/or
(e) is non-genetically engineered.

39-42. (canceled)

43. The oleaginous yeast strain of claim 38, said strain comprises an upregulation of:
(a) one, two, three, more or all of the genes as depicted in FIG. 7 and/or of the following genes:
(i) XM_016418757.1 (encoding zinc-binding oxidoreductase CipB);
(ii) XM_016418495.1 (encoding zinc-binding oxidoreductase ToxD);
(iii) XM_016417115.1 (encoding a gluconate 5-dehydrogenase);
(iv) XM_016421206.1 (encoding a gluconate 5-dehydrogenase); and
(v) XM_016417695.1 (encoding stress activated SIN1 from mitogen activated kinase (MAPK) superfamily);
(b) one, two, three or all of the following genes:
(vi) XM_016418081.1;
(vii) XM_016413927.1;
(viii) XM_016416917.1; and
(ix) XM_016420384.1;
(c) at least one, preferably both, of XM_016418757.1 and XM_016418495.1;
(d) at least one, preferably both, of XM_016418757.1 and XM_016413927.1.

44-46. (canceled)

47. The oleaginous yeast strain of claim 43, wherein said upregulation is:
(a) upregulation of gene expression on mRNA level;
(b) compared to a non-adapted control oleaginous yeast strain;
(c) compared to a control oleaginous yeast strain which was grown under fermentation conditions without the use of a lignocellulose-derived fermentable carbon source;
(d) compared to a control oleaginous yeast strain which was grown under pure glucose fermentation conditions;
(e) a magnitude of fold change of ≥2 as compared to a control oleaginous yeast strain; and/or
(f) tested by RNA expression profiling, transcriptomic sequencing and/or RNA quantification.

48-53. (canceled)

54. A method of producing oleaginous yeast cells, said method comprising
(I) the steps of
(i) growing oleaginous yeast cells in a medium which is essentially free of compounds which inhibit the growth of said oleaginous yeast cells and which are derivable from lignocellulosic material, wherein said medium comprises at least one carbon source which is fermentable by said yeast cells;
(ii) adding to said medium comprising said yeast cells a lignocellulosic preparation including one or more compound(s) which inhibit(s) the growth of said oleaginous yeast cells and which are derivable from lignocellulosic material, and further growing said oleaginous yeast cells in the medium;
(ii′) optionally inducing/increasing lipogenesis in said oleaginous yeast cells; and
(iii) harvesting said oleaginous yeast cells; or
(II) the steps of
(i) growing an oleaginous yeast strain of claim 38 in a lignocellulose medium;
(ii′) optionally inducing/increasing lipogenesis in said oleaginous yeast strain; and
(ii) harvesting the cells of said grown oleaginous yeast strain.

55. A method of producing fatty acids and/or lipids, said method comprising
(I) the steps of
(i) growing oleaginous yeast cells in a medium which is essentially free of compounds which inhibit the growth of said oleaginous yeast cells and which are derivable from lignocellulosic material, wherein said medium comprises at least one carbon source which is fermentable by said yeast cells;
(ii) adding to said medium comprising said yeast cells a lignocellulosic preparation including one or more compound(s) which inhibit(s) the growth of said oleaginous yeast cells and which are derivable from lignocellulosic material, and further growing said oleaginous yeast cells in the medium;
(ii′) optionally inducing/increasing lipogenesis in said oleaginous yeast cells;
(iii) harvesting said oleaginous yeast cells; and
(iv) extracting from said oleaginous yeast cells fatty acids and/or lipids; or
(II) the steps of
(i) growing the oleaginous yeast strain of claim 38 in a lignocellulose medium;
(ii′) optionally inducing/increasing lipogenesis in said oleaginous yeast strain;
(ii) harvesting the cells of said grown oleaginous yeast strain; and
(iv) extracting from said cells of said grown oleaginous yeast strain fatty acids and/or lipids.

56-59. (canceled)
